SEQUENCE LISTING (16 sequences)

SEQ ID NO: 1 - DNA, 20 bp, Artificial Sequence
  synthetic primer
tcactctccgagatggtggt

SEQ ID NO: 2 - DNA, 18 bp, Artificial Sequence
  synthetic primer
gtgcccgtggtcaattct

SEQ ID NO: 3 - DNA, 20 bp, Artificial Sequence
  synthetic primer
tctggccttccactctcagt

SEQ ID NO: 4 - DNA, 20 bp, Artificial Sequence
  synthetic primer
gactggcggggtgtaagtaa

SEQ ID NO: 5 - DNA, 18 bp, Artificial Sequence
  synthetic primer
gagggcttggttgtcagc

SEQ ID NO: 6 - DNA, 25 bp, Artificial Sequence
  synthetic primer
caattctcatggtagtgagttttcc

SEQ ID NO: 7 - DNA, 21 bp, Artificial Sequence
  synthetic primer
tgtatcgcaggcactcaggtc

SEQ ID NO: 8 - DNA, 25 bp, Artificial Sequence
  synthetic primer
ttcccactcgtttctggtagttctt

SEQ ID NO: 9 - DNA, 20 bp, Artificial Sequence
  synthetic primer
ggacacagaggtttcagtgg

SEQ ID NO: 10 - DNA, 20 bp, Artificial Sequence
  synthetic primer
ccagtagcaccatcatttcc

SEQ ID NO: 11 - DNA, 20 bp, Artificial Sequence
  synthetic primer
agctacggcaatcctgaact

SEQ ID NO: 12 - DNA, 20 bp, Artificial Sequence
  synthetic primer
gggccttctttacatttcca

SEQ ID NO: 13 - DNA, 21 bp, Artificial Sequence
  synthetic primer
agagttacgagttgcctgatg

SEQ ID NO: 14 - DNA, 22 bp, Artificial Sequence
  synthetic primer
ctgttgtaggtggtttcatgga

SEQ ID NO: 15 - DNA, 21 bp, Artificial Sequence
  synthetic primer
aacttcctacgtatgcacctg

SEQ ID NO: 16 - DNA, 20 bp, Artificial Sequence
  synthetic primer
aagtggctccattacgacag

(19) United States
(12) Patent Application Publication
(10) Pub. No.: US 2019/0290800 A1
(43) Pub. Date: Sep. 26, 2019

(54) POLYMERS AND USES THEREOF IN MANUFACTURING OF 'LIVING' HEART VALVES

(71) Applicants: CENTRO CARDIOLOGICO MONZINO, Milano (MI) (IT); THE UNIVERSITY COURT OF THE UNIVERSITY OF EDINBURGH, South Bridge Edinburgh (GB)

(72) Inventors: Maurizio PESCE, Roma (RM) (IT); Rosaria SANTORO, Cavenago di Brianza (MB) (IT); Mark BRADLEY, Edinburgh (GB); Seshasailam VENKATESWARAN, England (GB)

(21) Appl. No.: 16/085,662

(22) PCT Filed: Mar. 17, 2017
(86) PCT No.: PCT/EP2017/056357

§ 371 Date: Sep. 17, 2018

(30) Foreign Application Priority Data
Mar. 17, 2016 (EP) 16160995.3

(51) Int. Cl. A61L 27/16; A61L 27/58; A61L 27/18; A61L 27/26; A61L 27/54; A61L 27/34; A61L 27/38; C08F 220/34; C08F 220/28; C08F 220/32; C08F 220/14; D05C 17/00; D04H 3/011; B33Y 80/00; B33Y 70/00; D04H 3/007

(52) U.S. Cl. CPC A61L 27/16; D10B 2321/08; A61L 27/18; A61L 27/26; A61L 27/54; A61L 27/34; A61L 27/3873; A61L 27/3826; C08F 220/34; C08F 220/28; C08F 220/32; C08F 220/14; D05C 17/00; D04H 3/011; B33Y 80/00; B33Y 70/00; D04H 3/007; A61L 2430/20; A61L 2420/02; A61L 2300/64; A61L 2300/606; C08F 2220/281; C08F 2220/325; C08F 2800/10; D05D 2209/00; D10B 2509/06; D10B 2331/04; D10B 2401/12; A61L 27/58

(57) ABSTRACT

The present invention relates to a polymer having a first monomer selected from the group consisting of: styrene, MMA, HEMA or MEMA and a second monomer selected from the group consisting of: GMA, DEAEA, DEAEMA, DMAA, BAEMA, 4-vinylpyridine, DMVBA, 1-vinylimidazole, DMAEA or a combination thereof as coating agent for a scaffold or a medical device, to promote cellular adhesion and/or cell growth or for the manufacture of yarns or threads. The polymer may further contain a third monomer selected from the group consisting of: BMA, DEGMEMA, DAAA and MMA. The invention also relates to a scaffold, a medical device, a yarn, a thread or a textile coated or manufactured with the polymers of the invention and relative methods.

### TECHNICAL FIELD

The present invention relates to a polymer comprising a first monomer selected from the group consisting of: styrene, MMA, HEMA or MEMA and a second monomer selected from the group consisting of: GMA, DEAEA, DEAEMA, DMAA, BAEMA, 4-vinylpyridine, DMVBA, 1-vinylimidazole, DMAEA or a combination thereof as coating agent for a scaffold or a medical device, to promote cellular adhesion and/or cell growth or for the manufacture of yarns or threads. The polymer may further contain a third monomer selected from the group consisting of: BMA, DEGMEMA, DAAA and MMA. The invention also relates to a scaffold, a medical device, a yarn, a thread or a textile coated or manufactured with the polymers of the invention and relative methods.

### BACKGROUND ART

Diseased and dysfunctional heart valves are routinely repaired or replaced through surgical intervention. If damage is too severe to enable valve repair, the native valve is replaced by a prosthetic valve. About 300,000 heart valve procedures are performed annually worldwide and that number is expected to triple by 2050 with the majority of the patients over the age of 65. Commercially available heart valve prostheses are at present either mechanical or biological [1, 2]. Despite having excellent durability and a long-term mechanical performance, the mechanical prostheses are prone to thromboembolic complications causing patients to undergo lifelong anti-coagulation therapy. Biological valves, however, undergo structural leaflet deterioration. This is still the principal cause of prosthetic valve failure in the mid/long term, affecting a significant proportion of patients, especially in the young [3]. Deterioration of the biological implants is caused primarily by a chronic inflammatory condition resulting from a non-complete detoxification of the fixative remnants from the xenograft tissue [4, 5], or by the failure of the fixation protocols to remove major xenoantigens such as 1, 3 α-Galactose [6-10] (α-Gal). In addition, biological implants do not contain living cells, making them prone to infiltration by inflammatory elements of the recipient, that cause chronic inflammation.

The main feature of the natural valve leaflets is represented by the specific arrangement of the extracellular matrix (ECM) components (namely collagen, glycosaminoglycans and elastin), whose specific orientation and distribution in the thin leaflet width has uniquely evolved to result virtually in it being inextensible at valve closure during diastole and be soft and pliable to let the blood flow at valve opening during systole [11]. In order to fulfil these striking mechanical properties, the three dimensional structure of the valve tissue is extremely specialized. It is comprised of three layers with a different cellular and ECM composition that ensure correct absorption of the mechanical stress. In particular, the presence of anisotropically arranged collagen bundles in the fibrosa is the crucial structural component in ensuring the stress resistance of the leaflet at valve closure, while the presence of elastin in the ventricularis is specifically needed for the leaflet to recoil to its crimped initial state after diastolic loading [12-14]. The specific arrangement of collagen bundles determines the striking anisotropic mechanical characteristics of the valve tissue. In particular, this ensures a leaflet maximal stress resistance at the commissures and at the ‘belly’ portions, where the largest mechanical stresses are predicted, according to computational stress modelling.

The cellular composition and distribution in the valve is also specialized with external valve endothelial cells (VECs) lining inflow and outflow valve surfaces. It also has valve interstitial cells (VICs), cells with a plastic fibroblast/myofibroblast phenotype, which provide the necessary renewal of ECM components for a tissue undergoing 3 billion load/unload cycles in its average lifetime, [15]. It has been discussed that the mechanical forces, especially during the embryonic shaping of the heart valves, give a primary contribution to differentially align and determine different shapes of VECs on the two leaflet surfaces, and are crucial to induce differential strain-dependent maturation of the valve fibrillar matrix structure by modulating the function/phenotype of VICs in the three presumptive layers (reviewed in [16]).

Despite past and ongoing intense efforts to mimic the mechanical and the biological features of the uniquely specialized valve tissue by an artificial tissue produced in a single manufacturing process, definitive solutions are still awaited. Even the most advanced approaches available today do not reproduce the structural features and the cell/materials interactions to grant tissue stability and endure cyclic strains throughout an entire lifetime. In our view this challenge requires a novel approach to design a ‘VIC-containing’ and mechanically stable valve made of a ‘cell-instructing’ polymer that promotes valve homeostasis by modulating the biological interactions between the artificial microenvironment and VICs; this will finally overcome the current shortcomings in tissue engineered heart valves (TEHVs).

Apart from developing novel manufacturing improvements to ameliorate the performance of the mechanical valves or the durability of the biological/bioprosthetic valves (implantable by mini-invasive or trans-catheter procedures) [2], numerous possibilities have been proposed to design optimized replacement valve implants that may be used as alternative to the currently employed devices. These approaches have led to two alternative manufacturing processes leading to the design of: i) prostheses made completely of artificial materials (i.e. polyurethanes) providing an optimal mechanical resistance along with a surface/material functionalization to limit the coagulation risk typical of the mechanical valves (the so-called polymeric valves; PVs) [17, 18] or of, ii) implants manufactured by combining 3D-printed, electrospun, or multi-layered biodegradable scaffolds with living cells (the so-called tissue engineered heart valves; TEHVs) (reviewed in [19]). The advantages and the potential shortcomings of each of these two approaches have been well described elsewhere. Here it will be sufficient to mention that, so far, none of these alternatives have led to marketable valve prostheses that may benefit from the main advantages of the PVs (design of leaflets with mechanically controlled performance, maintenance of leaflet geometry) and those of TEHVs (presence of living cells depositing ECM components, potential to self-renew) and, at the same time, avoid shortcomings such as an insufficient anti-coagulation in the long term or the propensity to calcification, typical in PVs [17, 18], or the propensity to increase thickness or to show ‘retraction’ or ‘compaction’ known to compromise the performance of TEHVs based on bio-absorbable polymer technology [19, 20].

The development of new technologies that improve the quality of the therapies in heart valve replacement is expected to have an enormous impact on reduction of economic and social costs of cardiac valve pathologies. In fact, the invention of new materials and processes to produce a totally biocompatible valve tissue may open novel perspectives for improved implant quality, duration and performance, which may turn into higher quality of life for patients and new marketing opportunities. The two alternatives to surgeons to implant artificial valves are, in fact, represented by mechanical and bio-prosthetic devices, that in both cases, have major contra-indications. These consist in the need to treat patients with a continuous anticoagulation therapy in the case of mechanical valves, or in the limited durability of the animal derived tissue, normally bovine pericardium and porcine valves, used to manufacture the bioprosthetic valve implants. For these reasons, on top from the costs sustained by the Health Systems for hospitalization of patients who need valve replacement, the costs for patients everyday management as well the impact on quality of life are unacceptably high, especially in case of pediatric patients. While the surgical replacement of diseased valves is overall evolving toward mini-invasive or trans-catheter procedures, few comparable advancements have been made toward the manufacture of living bio-valve implants with an acceptable life-time without the side effects of the current TEHVs. According to these considerations, the introduction of a radically new technology in this field is urgently needed to offer patients, especially the young, a novel generation of ‘off-the-shelf’ valve bio-implants carrying, at the same time, the mechanical performance of the natural valves and the ability of the engineered tissues to self-renew, to last for a long time, and adapt to the recipient's biological environment. Morsi Y S. Bioengineering strategies for polymeric scaffold for tissue engineering an aortic heart valve: an update, Int J Artif Organs 2014; 37(9): 651-667, highlight the bioengineering strategies that need to be followed to construct a polymeric scaffold of sufficient mechanical integrity, with superior surface morphologies, that is capable of mimicking the valve dynamics in vivo. The current challenges and future directions of research for creating tissue-engineered aortic heart valves are also discussed.

Claiborne T E et al. Polymeric trileaflet prosthetic heart valves: evolution and path to clinical reality. Expert Rev Med Devices. 2012 November; 9(6): 577-594, review the evolution of Polymeric heart valves (PHVs), evaluate the state of the art of this technology and propose a pathway towards clinical reality. In particular, the authors discuss the development of a novel aortic PHV that may be deployed via transcatheter implantation, as well as its optimization via device thrombogenicity emulation.

WO2012/172291 relates to the use of certain polymers as a substrate for stem cell, such as pluripotent stem cell growth and/or culture, and to articles such as tissue culture materials and cell culture devices comprising at least one polymer hydrogel.

WO2010/023463 refers to a biocompatible polymer mixture for use as a matrix for cellular attachment including a mixture of at least two polymers selected from the group consisting of: chitosan (CS), polyethylenimine (PEI), poly (L-lactic acid) (PLLA), poly (D-lactic acid) (PDLA), poly (2-hydroxy ethyl methacrylate) (PHEMA), poly (e-caprolactone) (PCL), poly(vinyl acetate) (PVAc), poly (ethylene oxide) (PEO), poly [(R)-3-hydroxybutyric acid)] (PHB), cellulose acetate (CA), poly (lactide-co-glycolide) (PLGA) and poly (N-isopropylacrylamide) (PNIPAM). Implants making use of the polymer mixtures can support cell attachment, growth and differentiation, and tissue regeneration in vivo.

WO2006016163 refers to polymers suitable for use as medical materials and to polymer useful as a medical material having the general formula: (I)-(A)l-(B)m-(C)n- in which A is derived from an alkoxyalkyl (alkyl)acrylate monomer; B is derived from a monomer containing a primary, secondary, tertiary or quaternary amine group; C is derived from a non-ionic monomer; and 1+m+n=l00, 0<l, m, n<l00.

WO2014/170870 refers to a prosthetic heart valve which includes a stent having three leaflets attached thereto.

WO2014143498 relates to a thin, biocompatible, high-strength, composite material that is suitable for use in various implanted configurations. The composite material maintains flexibility in high-cycle flexural applications, making it particularly applicable to high-flex implants such as for myocardium or heart valve leaflet reconstructions. The composite material includes at least one porous expanded fluoropolymer layer and an elastomer filling the porous expanded fluoropolymer.

WO2014008207 refers to a prosthetic heart valve including a base and a plurality of polymeric leaflets.

US2013325116 refers to a prosthetic heart valve including annularly spaced commissure portions, each of which includes a tip. The valve stent is manufactured with a polymeric material, and is specifically configured to perform similarly to conventional metal stents. CN102670332 relates to an artificial heart valve which is implanted to replace a dysfunctional heart valve by surgical operation or vascular intervention. The artificial heart valve comprises a stent and a valve leaflet.

US2014303724 refers to a polymeric valve which may include a heart valve, and also may include a leaflet heart valve including a stent having a base and a plurality of outwardly extending posts from the base and equidistant from each other.

WO2011/130559 refers to a polymeric heart valve including: a valve body having a central axis having a body fluid pathway extending along the central axis from an inflow end to an outflow end; a flexible stent disposed about an outer circumference of the body and including at least three flexible stent posts each extending in the axial direction to a tip; and at least three flexible leaflets extending from the stent, each of the leaflets having an attached edge defining an attachment curve along the stent extending between a respective pair of stent posts.

WO2008045949 relates to a bioprosthetic heart valve having a polyphosphazene polymer such as poly[bis(trifluoroethoxy)phosphazene], which exhibits improved antithrombogenic, biocompatibility, and hemocompatibility properties. A method of manufacturing a bioprosthetic heart valve having a polyphosphazene polymer is also described.

WO2007062320 refers to a prosthetic heart valve that includes three leaflet members which open and close in unison with the flowing of blood through the aorta. The leaflets are made of a composite multilayer polymer material that includes a central porous material such as polyethylene terephthalate sandwiched between two other polymer layers.

WO2007013999 refers to a Catheter Based Heart Valve (CBHV) which replaces a non-functional, natural heart valve. The CBHV significantly reduces the invasiveness of the implantation procedure by being inserted with a catheter as opposed to open heart surgery. Additionally, the CBHV is coated with a biocompatible material to reduce the thrombogenic effects and to increase durability of the CBHV. The CBHV includes a stent and two or more polymer leaflets sewn to the stent. The stent is a wire assembly coated with Polystyrene-Polyisobutylene-Polystyrene (SIBS). The leaflets are made from a polyester weave as a core material and are coated with SIBS before being sewn to the stent.

In WO2006000776, implantable biocompatible devices such as synthetic prosthetic heart valves are disclosed. The leaflet aortic heart valve design has three valve leaflets supported on a frame.

WO2005049103 relates to a heart valve sewing prosthesis including an intrinsically conductive polymer.

US2003114924 refers to a prosthetic heart valve comprising a valve body and a plurality of flexible leaflets. Each leaflet comprises an attachment end, anchored to the valve body, and a free margin.

DE19904913 relates to a flexible polymer heart valve for replacement of a human heart valve which is modified by a plasma process.

U.S. Pat. No. 5,562,729 refers to a multi-leaflet (usually trileaflet) heart valve composed of biocompatible polymer which simultaneously imitates the structure and dynamics of biological heart valves and avoids promotion of calcification.

WO9714447 refers to a biomaterial such as a synthetic polymer, metal or ceramic and a therapeutically effective amount of Triclosan used in the manufacture of medical devices or prostheses for internal or in vivo medical applications. Medical devices or prostheses containing such biomaterials are also disclosed, including prosthetic hip and knee joints, artificial heart valves, voice and auditory prostheses.

KR930002210 relates to a modified polymeric material with improved blood compatibility that is obtained by substituting the amide or acid amide groups of a polymeric substrate with a sulfonated polyethylene oxide (PEO) groups.

WO8900841 relates to a protective shield which covers the sewing cuff and sutures of implanted prosthetic heart valves. The protective shield is made from, or coated with, a material that is bio and blood-compatible and non-thrombogenic, such as polished pyrolytic carbon or acetal polymer.

ES8406873 refers to device, in particular a cardiac valve prosthesis having elements at least partly formed of polymer or a vascular prosthesis with a tubular body of polymeric textile material, has a coating of biocompatible carbonaceous material.

GB1270360 refers to a prosthetic heart valve having four closure flaps.

In WO2005097227, a composition is disclosed comprising a structural component comprising linear acrylic homopolymers or linear acrylic copolymers and a bio-beneficial component comprising copolymers having an acrylate moiety and a bio-beneficial moiety.

In WO2006036558, a polymer for a medical device, particularly for a drug eluting stent, is described. The polymer can be derived from n-butyl methacrylate and can have a degree of an elongation at failure from about 20% to about 500%.

In CN101361987, a heart valve prosthesis suture ring of terylene with an antibacterial function is provided as well as a preparation method thereof.

FR2665902 refers to new polymers substituted with sulphonated polyethylene oxide which have an improved blood compatibility. They are obtained by substitution of a polymer substrate which has active sites of amide groups or acid amide groups, such as a polyurethane, a polyamide and a polyacrylamide, with sulphonated polyethylene oxide [PEO-(SO3H)n]. The polymers are valuable as materials of construction for artificial organs for the circulatory system, which are intended to be in contact with blood, such as artificial hearts, artificial blood vessels, artificial kidneys and the like. GB1159659 describes medical and dental devices and tissue implants for use in contact with blood having on the surface carboxyl groups which render the surface of the device anti-coagulative when in contact with blood.

### SUMMARY OF THE INVENTION

Compared with already existing technologies like those based on electrospinning of bio-absorbable materials, inventors focused their attention on polymers largely based on acrylates which due to their chemistry flexibility may be employed as:

i) a coating material able to functionalize preformed scaffold to instruct correct differentiation of heart valves-derived cells for tissue engineering applications;

ii) basic material to obtain fibers and yarns with specific mechanical/biological features;

iii) embroidery material to generate textile-like scaffolds recapitulating the mechanical properties of the natural valve leaflets.

The inventors identified non-bioabsorbable materials, whose adjustable mechanical features and biological functionalization are very versatile for the manufacture of cellularized biological implants, leading to a 3D scaffold manufacturing process based on fibre coating, spinning and embroidery technologies.

In the present patent application, the inventors claim the identification of such novel materials tailored for culturing heart valve interstitial cells (VICs). These materials have been identified by a high-throughput screening approach (polymer arrays), followed by assessment of their biological compatibility in cell culture, and translation into a 3D environment by bioreactor-assisted VICs seeding.

The identified polymers provide a new class of non-biodegradable VICs-tested materials for manufacturing off-the-shelf tissue engineered valve (TEHV) prostheses. Novel materials may be tailored for culturing heart valve interstitial cells (VICs).

The present invention provides the use of at least one polymer comprising:
a first monomer selected from the group consisting of: styrene, MMA, HEMA or MEMA and
a second monomer selected from the group consisting of: GMA, DEAEA, DEAEMA, DMAA, BAEMA, 4-vinylpyridine, DMVBA, 1-vinylimidazole, DMAEA as coating agent for a scaffold or a medical device.

The present invention provides the use of at least one polymer comprising:
a first monomer selected from the group consisting of: styrene, MMA, HEMA or MEMA and
a second monomer selected from the group consisting of: GMA, DEAEA, DEAEMA, DMAA, BAEMA, 4-vinylpyridine, DMVBA, 1-vinylimidazole, DMAEA to promote cell adhesion and/or cell growth.

The present invention provides the use of at least one polymer comprising:
a first monomer selected from the group consisting of: styrene, MMA, HEMA or MEMA and
a second monomer selected from the group consisting of: GMA, DEAEA, DEAEMA, DMAA, BAEMA, 4-vinylpyridine, DMVBA, 1-vinylimidazole, DMAEA for the manufacture of yarns or threads.

The polymer may further comprise a third monomer selected from the group consisting of: BMA, DEGMEMA, DAAA or MMA.

Every combinations of the above monomers is comprised within the present invention. Preferably the polymer is selected from a polymer comprising:
styrene and DMAA,
MMA and GMA or DEAEMA or DMAEA,
MEMA and DEAEA or DEAEMA or BAEMA or 4-vinylpyridine or GMA,
HEMA and BAEMA or DMVBA or 1-vinylimidazole or 4-vinylpyridine.

Preferably the ratio between the first monomer and the second monomer is between 40:60 and 90:10. Still preferably the ratio between the first monomer and the second monomer is between 50:50 and 90:10. The preferred ratio between the first monomer and the second monomer are 50:50, 90:10, 70:30, 55:45.

In a preferred embodiment, the ratio between the first monomer, the second monomer and the third monomer is between 40:30:30 and 60:30:10.

Preferably, the polymer is functionalized. Preferably, the functionalization is carried out by an amine or a thiol. In particular, polymers containing the GMA monomer are amine or thiol functionalized. Preferably the functionalization is carried out by an amine selected from the group consisting of: DnHA, DBnA, TEDETA, Mpi, TMPDA, DEMEDA, TMEDA, Pyrle, MAEPy, BnMA, MnHA, DcHA, cHMA, MAn, DnBA and DnHA.

In a preferred embodiment the polymer is PA6, PA98, PA309, PA316, PA317, PA321, PA338, PA426, PA438 (Ranked with a score 3 according to screening results), PA104, PA111, PA112, PA134, PA167, PA176, PA181, PA187, PA255, PA285, PA295, PA296, PA318, PA319, PA324, PA326, PA329, PA354, PA364, PA506, PA512, PA516 or PA531 (Ranked with a score 2 according to screening results) as defined in Table I.

Another object of the invention is the at least one polymer as above defined, for use in a method to promote in vivo cell adhesion and/or in vivo cell growth. Said method is preferably performed with a scaffold or a medical device coated with or comprising (or consisting of) the said at least one polymer, or with yarns or threads manufactured with said at least one polymer or with textile manufactured with said yarn or thread.

Preferably the medical device is implantable or the scaffold is bio-absorbable.

Still preferably the medical device consists of a device selected from the group of: heart valve substitute, heart valve implant, heart valve bio-artificial tissue, heart valve tissue scaffold, preferably a tissue engineered heart valve (TEHV) prosthesis.

Preferably, the medical device comprises (or consists of) polycaprolactone.

In a preferred embodiment the cell is a cell type with the characteristics of mesenchymal cell such as: bone marrow-derived mesenchymal cells, cardiac-derived mesenchymal cells, cardiac-derived fibroblasts, pericyte-derived mesenchymal cells, cord blood-derived mesenchymal cells, placental-derived mesenchymal cells, induced Pluripotent Stem Cells, Vascular-derived progenitor cells, Endothelial (Progenitor) cells, heart valve interstitial cells, preferably the cells are aortic/mitral valve interstitial cell.

The present invention provides a scaffold or a medical device coated with or comprising (or consisting of) the polymer as defined above. Preferably the medical device is a tissue engineered heart valve (TEHV) prosthesis. In a preferred embodiment the scaffold or medical device is for use in a surgical method or a minimally invasive implantation procedure. The present invention provides a yarn or a thread manufactured with the polymer as defined above. The present invention provides a textile manufactured with the yarn or thread as defined above. The scaffold or medical device, the yarn or thread or the textile as above defined, may further comprise:

a) living cells produced by in vitro incubation and/or

b) additional components selected from the group consisting of growth factors, DNA, RNA, proteins, peptides and therapeutic agents for treatment of disease conditions wherein said cells are attached to the polymer. The scaffold or medical device, the yarn or thread, the textile as above defined are preferably for use in a surgical method, preferably for use in the repair or replacement of living tissue.

The present invention provides method to coat a scaffold or a medical device with the polymer as defined above comprising coating said scaffold or medical device by a method selected from the group consisting of: grafting, dipping, spraying, electrospinning, 3D printing or other methods known to those skilled in the art.

The present invention provides a method to manufacture the textile as defined above comprising electrospinning and/or embroidery.

A further object of the invention is a method for repair or replacement of tissue comprising: providing the scaffold or medical device, the yarn or thread or the textile as above defined, and locating the said scaffold or medical device or yarn or thread or textile on or in the body of a subject.

Any combination of the polymers according to the invention is included in the present invention.

“Culturing” as used herein refers to the growth, maintenance, storage and passaging of cells. Cell culture techniques are well understood and often involve contacting cells with particular media to promote growth. In the present case, cells contacted with or exposed to polymer of the present invention during culture may continue to grow and/or proliferate and/or differentiate. The base-substrate of the polymer of the invention may be a solid or semi-solid substrate. Suitable examples may include base-substrates comprising, for example, glass, plastic, nitrocellulose or agarose. In one embodiment, the base-substrate may take the form of a glass or plastic plate or slide. In other embodiments, the base-substrate may be a glass or plastic multi-well plate such as, for example a micro-titre plate. In one embodiment the base-substrate may take the form of a tissue culture flask, roller flasks or multi-well plate. The base-substrate may be coated with the polymer of the invention. The base-substrate may be coated with a layer or several layers of the polymer. The polymer of the invention may be incorporated into the main body of the substrate. The polymers of the present invention find particular application in cell culture products designed to facilitate the culture of cells, as e.g. pluripotent stem cells or mesenchymal cells. The polymers may be used for culturing cells in vitro. The polymers may form part of a tissue culture substrate. The polymers may be used to coat the base-surface of tissue culture substrates such as the base-surface of microtitre plates, cell culture flasks, roller flasks and the like. Typically only a base-surface which comes into contact with cells need be coated. Thus, the invention also provides a cell culture device or apparatus for use in the culture of cells, such as pluripotent stem cells, comprising at least one polymer as above defined and a base-substrate. The tissue culture apparatus may be pre-seeded with the cells or the apparatus may be ‘naked’ i.e. there may be no cells present. The tissue culture apparatus may comprise a growth medium to support cell culture. The tissue culture apparatus may comprise nutrients, antibiotics and other such additives to support cell culture. The implant (which may be a scaffold or medical device or yarn or thread or textile as above defined) may include living cells attached to the polymer of the invention. For example vascular-derived progenitor cells, heart valve interstitial cells, adult human bone marrow-derived skeletal stem/progenitor cells, human fetal skeletal progenitor cells or human articular chondrocytes. Alternatively the implant may be incubated with suitable cells, in vitro, prior to use, to provide an implant comprising tissue, which may be natural tissue or modified or genetically engineered natural tissue. Alternatively the implant may be used without attached cells or tissue whereupon it may be colonized by the subject's own cells, providing a matrix or scaffold for growth of the cells. Tissues that may be repaired or replaced by the implant of the invention include bone or cartilage. Other tissues, for example soft tissues such as muscle, skin or nerve may also be repaired or replaced. The implant may simply consist of at least one polymer of the invention with or without attached cells. Other components may be included in the implant. For example, the implant may include DNA, RNA, proteins, peptides or therapeutic agents for treatment of disease conditions. The implant may also include biodegradable and non-biodegradable components. For example, the implant may be a stent of a manufactured non-biodegradable material but coated with a selected polymer of the invention and optionally seeded with appropriate cells. For further example, the implant may be used for replacement of bone and may include a permanent support such as a steel plate or pin and a portion made from at least one polymer of the invention and seeded with bone producing cells. In use the steel plate or pin remains as a structural support, whilst the polymer mixture acts as a scaffold but degrades following the desired growth of bone tissue. The implants of the invention may be used to effect tissue repair or replacement. The invention therefore also provides a method for repair or replacement of tissue comprising: providing an implant as above defined; and locating the implant on or in the body of a subject. For example, the implant may be placed on the body of a subject when skin tissue is being repaired. For further example, the implant may be placed within a subject when bone or an internal organ is being repaired.

The present invention will be described through non-limitative examples, with reference to the following figures:

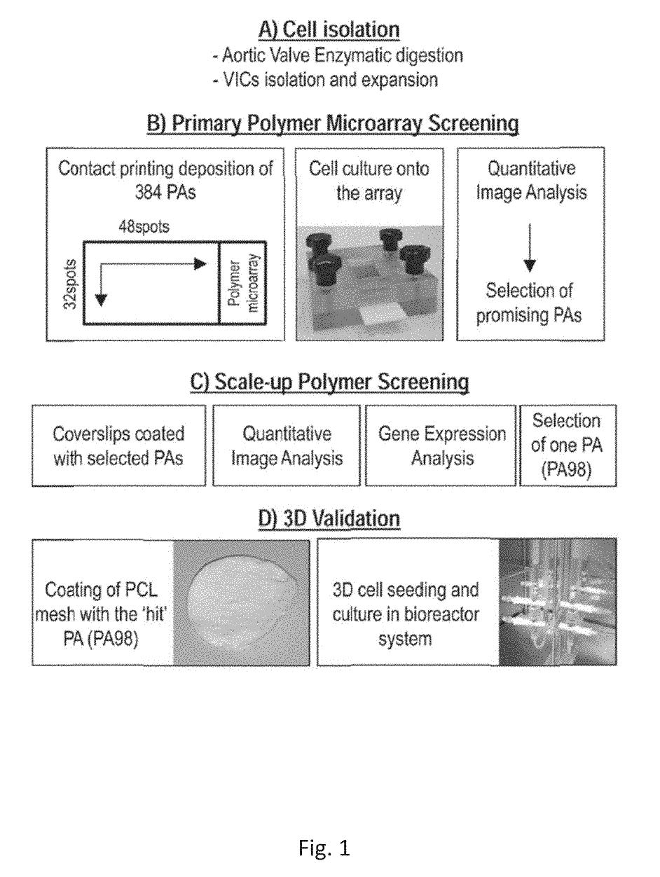

FIG. 1. Experimental flowchart illustrating the main actions performed to derive valve interstitial cells from the human AoV (A), to manufacture the polymer arrays to perform the primary screening (8), to scale up the identified ‘hits’ (C), and to transfer the PA98 in the 3D environment by the use of a perfusion system allowing dynamic cell seeding into a PA98-coated PCL scaffold (D).

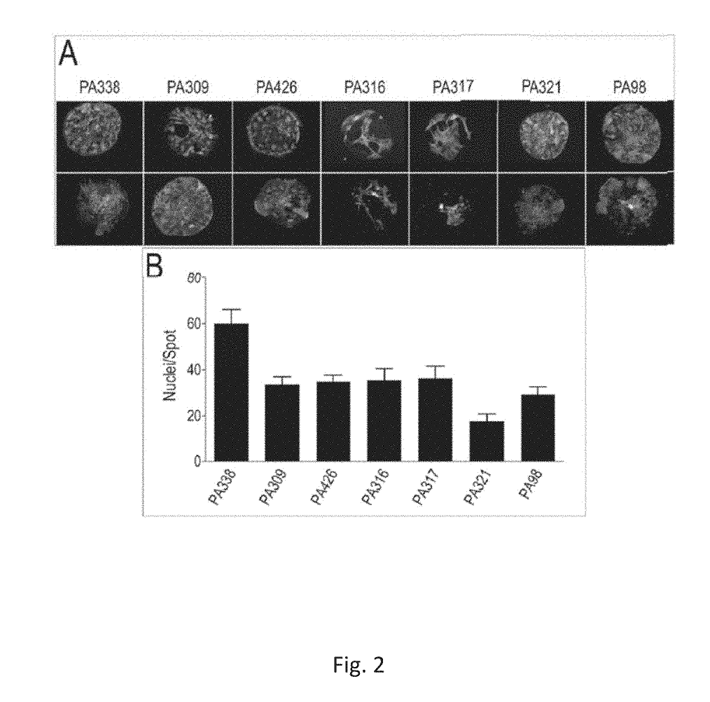

FIG. 2. Results of the primary polymer array screening. Panel A indicates two representative images of each spot of the ‘hit’ polymers covered by cells and stained with DAPI for nuclear staining (Blue fluorescence), with phalloidin-TRITC for actin stress fibers (Red fluorescence), and antibodies recognizing α-smooth muscle actin (Green fluorescence). Panel B shows cellular quantification/spot for each of the identified ‘hit’ PAs, based on nuclei counting.

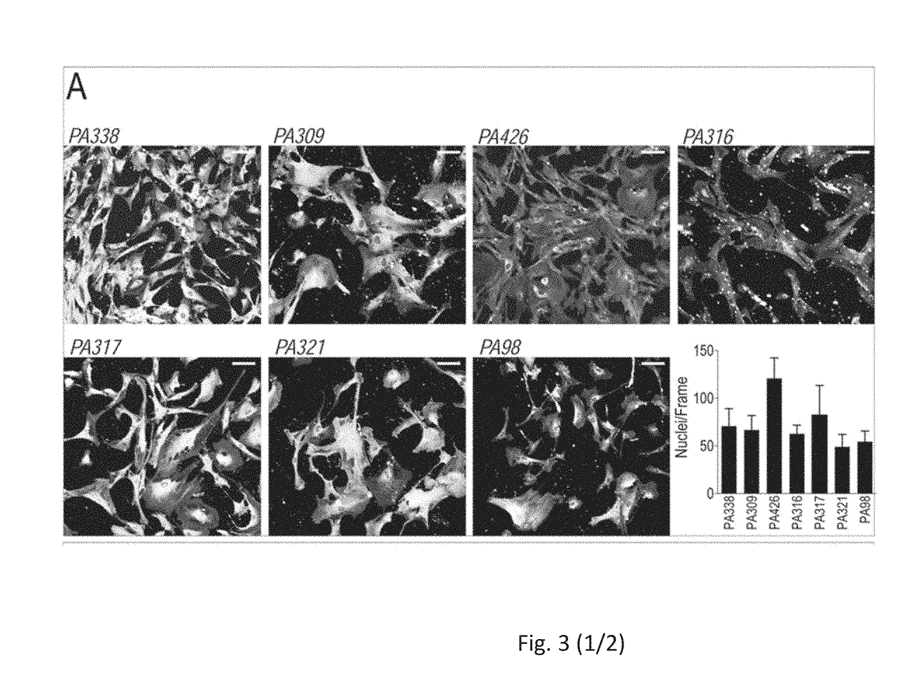

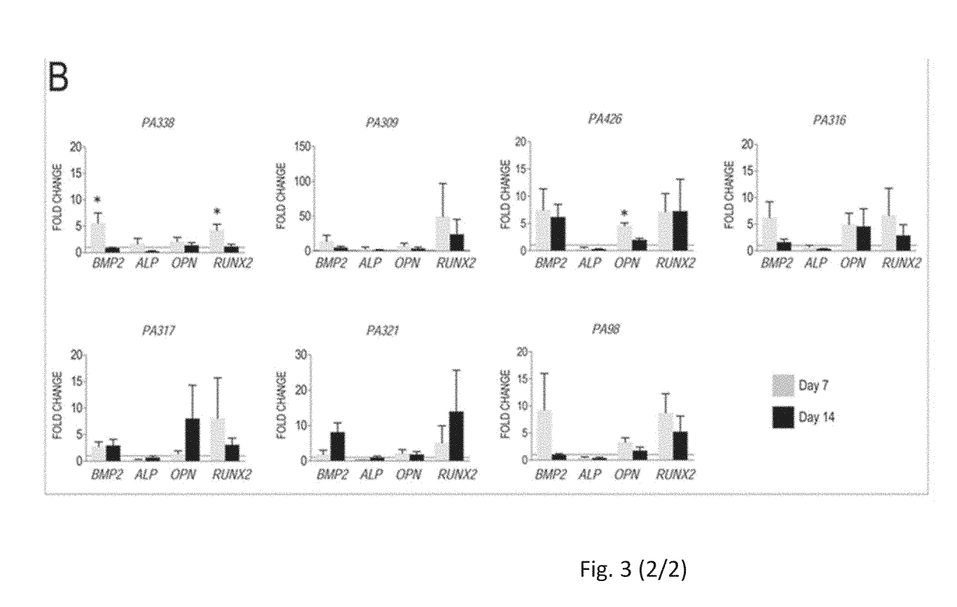

FIG. 3: Secondary screening of hit polymers on spin-coated glass slides. Panel A indicates an immunofluorescence staining for α-smooth muscle actin (Green fluorescence) and Collagen I (white fluorescence), in conjunction with nuclear staining (blue fluorescence) and stress fibers by phalloidin-TRITC staining (red fluorescence). The bar graph shows the number of cells attached to the polymer. (B) To show regulation of genes potentially involved in VICs pathologic differentiation, expression of genes involved in osteogenesis was analyzed by q-RT-PCR amplification in cells cultured on each of the selected polymers. Results are expressed as fold change gene expression in VICs cultured onto each of the polymers for the indicated periods vs. the expression level observed in the cells before the beginning of the culture (reference line at fold change=1). * indicates P<0.05 in the comparison day 7 vs. day 14 by unpaired Student's t-test (n≥4).

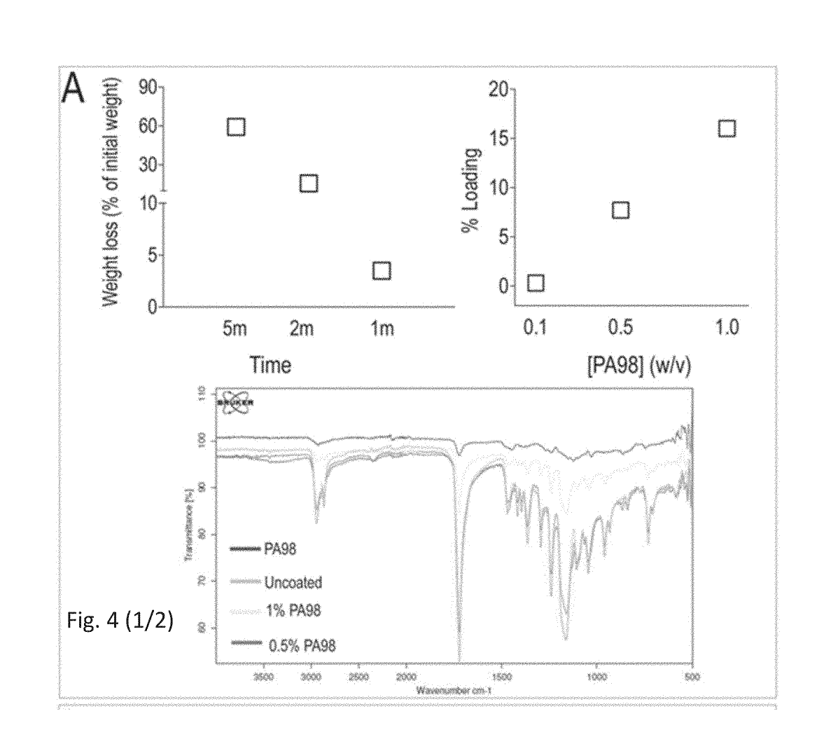

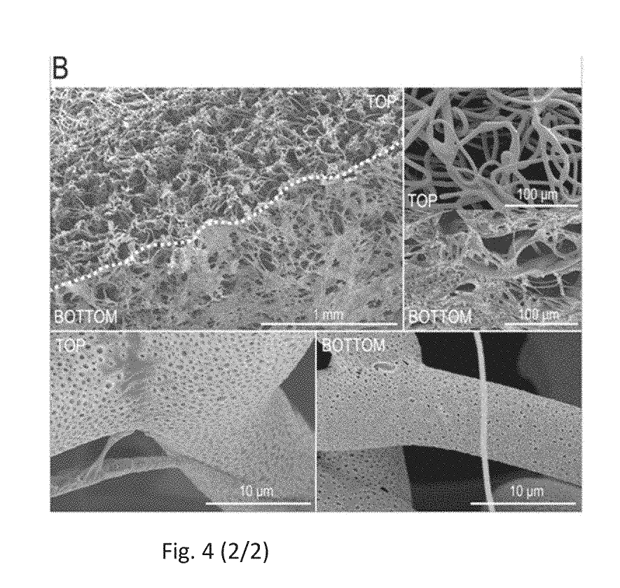

FIG. 4: (A) The graphs on the top indicate a time course of PCL scaffold weight loss caused by dipping into Acetone (left) and PA98 loading following incubation into solutions with increasing PA98 concentrations. IR spectra confirmed coating of PA98 on PCL scaffolds (dipping time approx. 1 sec). (B) Scanning Electron Microscopy images of the PCL scaffold loaded with 1% (w/v) PA98 solution. The coating procedure preserved the PCL porous structure.

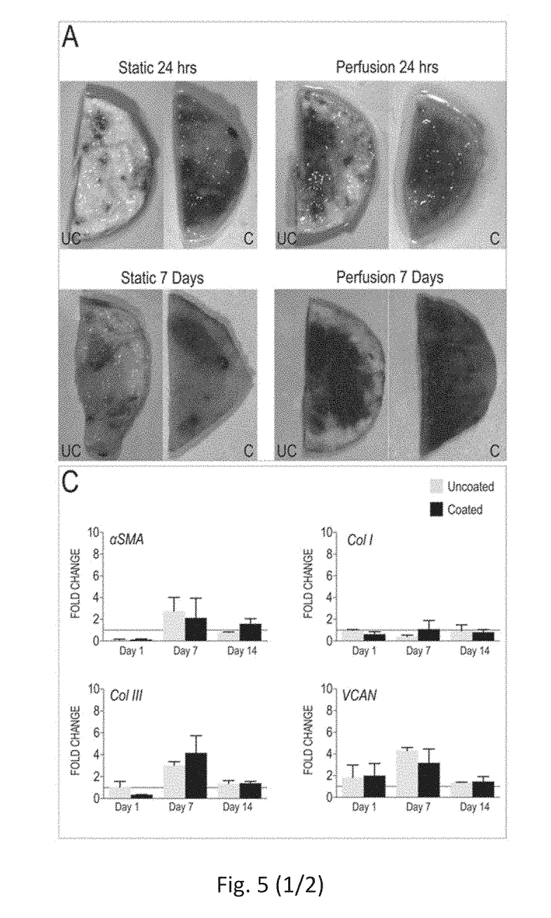

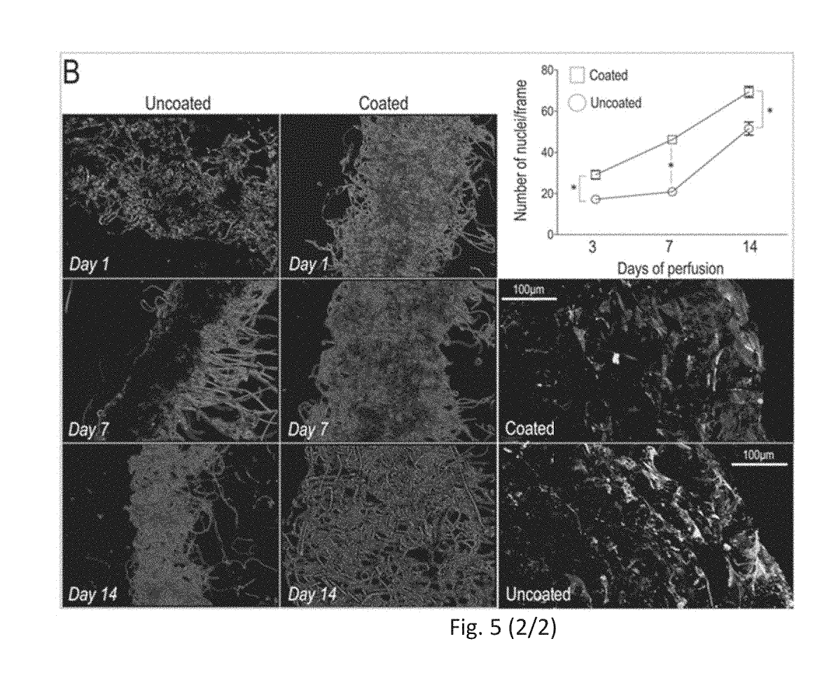

FIG. 5: Characterization of the 3D scaffolds after static or dynamic VICs seeding into a perfusion bioreactor system. (A) MTT staining of the un-coated (UC) and Polymer G-coated (C) PCL scaffolds seeded with the cells with or without perfusion for 24 hrs and 7 days. Results clearly indicate a higher efficiency in cell retention into the coated/perfused scaffolds in comparison with the other conditions. (B) Cell quantification by nuclear counting of cells per microscopic frame in transversal sections of dynamically seeded un-coated and coated scaffolds at the various time points. Analysis by 2-ways ANOVA with Bonferroni post-hoc test indicated a P<0.01 statistical significance in the difference between the number of cells in PA98-coated vs. uncoated scaffolds at all times. The insert shows a confocal microscopy image of sections of PA98-coated and un-coated scaffold seeded with VICs and stained with phalloidin-TRITC (red fluorescence) and α-smooth muscle actin (green fluorescence). (C) Expression analysis of α-Smooth Muscle Actin, Collagen I/111 and Versican genes in 14 days 3D cultured VICs in the indicated conditions. Results are expressed as fold change gene expression in VICs cultured in PA G-coated and uncoated vs. the expression level observed in the cells before the beginning of the culture (reference line at fold change=1).

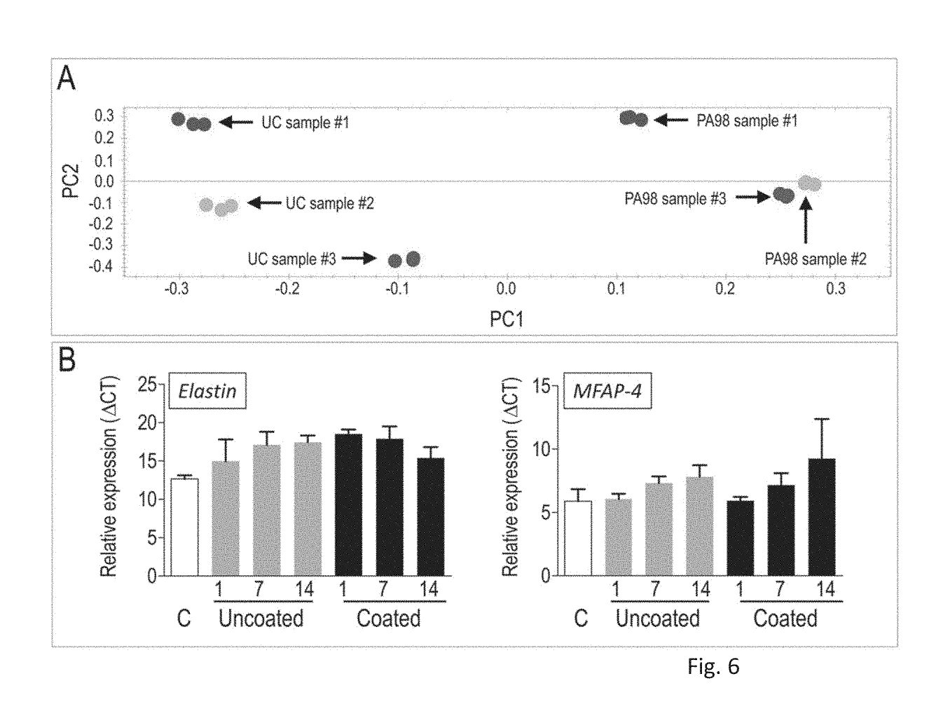

FIG. 6: Results of mass-spec analysis of proteins released by human VICs in uncoated or PA98-coated PCL scaffolds. (A) Principal component analysis of differentially expressed proteins in the three VICs seeded uncoated and PA98-coated scaffolds. As shown, a clear separation of each of the three technical replicates for each aortic VICs samples was found between the coated (PA98) and uncoated (UC) scaffolds, highlighting a fundamentally different release of extracellular proteins. (B) Real Time PCR amplification of Elastin and MFAP-4 mRNAs from VICs cultured into the uncoated and PA98-coated PCL scaffolds. Data are expressed as relative expression data based on delta-CT values to show the variation level of transcripts in cells in the initial cellular population vs. those present in the scaffolds at the different time points. Statistical analysis by 1 way Anova with Tukey post-hoc did not reveal significant differences of the Elastin and MFAP-4 mRNAs, suggesting that the increase in MFAP-4 protein expression in PA98-coated scaffold is mainly due to a post-translational process.

### DETAILED DESCRIPTION OF THE INVENTION

### Experimental Procedures

### Polymer Microarrays Preparation

Glass slides were soaked in 1M NaOH for 4 hours and cleaned thoroughly with distilled water. The cleaned slides were rinsed with acetone to remove the water and dried in ambient conditions before immersing in acetonitrile (15 mL) containing 1% (3-aminopropyl)triethoxysilane for 2 hours. Subsequently, the slides were cleaned with acetone (3×15 ml) and then placed into an oven (100° C.) for 1 hour. The aminosilane treated slides were collected and dip-coated with 1% agarose aqueous solution under 60° C. The agarose coated slides were left in ambient conditions for 24 hours before drying in a vacuum oven (45° C.) overnight. Polymer microarrays, containing 384 polymers (Table I), each printed in quadruplicate, were fabricated as previously reported [21].

TABLE 1
Polymer identity, monomer composition and results of the array screening

| | | | | Ratio (mol) | | | | |
|---|---|---|---|---|---|---|---|---|
| PA number | Monomer (1) | Monomer (2) | Monomer (3) | M (1) | M (2) | M (3) | Funct | Rank |
| 1 | St | DEAA | | 90 | 10 | | | 0 |
| 4 | St | DMAA | | 90 | 10 | | | 0 |
| 5 | St | DMAA | | 70 | 30 | | | 0 |
| 6 | St | DMAA | | 50 | 50 | | | 3 |
| 7 | St | PAA | | 90 | 10 | | | 0 |
| 8 | St | PAA | | 70 | 30 | | | 0 |
| 9 | St | PAA | | 50 | 50 | | | 1 |
| 10 | MMA | DEAA | | 90 | 10 | | | 1 |
| 11 | MMA | DEAA | | 70 | 30 | | | 1 |
| 12 | MMA | DEAA | | 50 | 50 | | | 1 |
| 13 | MMA | DMAA | | 90 | 10 | | | 0 |
| 14 | MMA | DMAA | | 70 | 30 | | | 0 |
| 15 | MMA | DMAA | | 50 | 50 | | | 0 |
| 16 | MMA | PAA | | 90 | 10 | | | 0 |
| 17 | MMA | PAA | | 70 | 30 | | | 1 |
| 19 | MEMA | DEAA | | 90 | 10 | | | 0 |
| 20 | MEMA | DEAA | | 70 | 30 | | | 0 |
| 21 | MEMA | DEAA | | 50 | 50 | | | 0 |
| 22 | MEMA | DMAA | | 90 | 10 | | | 1 |
| 23 | MEMA | DMAA | | 70 | 30 | | | 1 |
| 24 | MEMA | DMAA | | 50 | 50 | | | 0 |
| 25 | MEMA | PAA | | 90 | 10 | | | 0 |
| 26 | MEMA | PAA | | 70 | 30 | | | 0 |
| 27 | MEMA | PAA | | 50 | 50 | | | 0 |
| 28 | MEA | DEAA | | 90 | 10 | | | 0 |
| 30 | MEA | DEAA | | 50 | 50 | | | 0 |
| 31 | MFA | DMAA | | 90 | 10 | | | 0 |
| 32 | MEA | DMAA | | 70 | 30 | | | 0 |
| 33 | MEA | DMAA | | 50 | 50 | | | 0 |
| 34 | MEA | PAA | | 90 | 10 | | | 0 |
| 35 | MEA | PAA | | 70 | 30 | | | 0 |
| 37 | HEMA | DEAA | | 90 | 10 | | | 0 |
| 38 | HEMA | DEAA | | 70 | 30 | | | 0 |
| 39 | HEMA | DEAA | | 50 | 50 | | | 0 |
| 40 | HEMA | DMAA | | 90 | 10 | | | 0 |
| 41 | HEMA | DMAA | | 70 | 30 | | | 0 |
| 42 | HEMA | DMAA | | 50 | 50 | | | 0 |
| 43 | HEMA | PAA | | 90 | 10 | | | 0 |
| 44 | HEMA | PAA | | 70 | 30 | | | 0 |
| 45 | HEMA | PAA | | 50 | 50 | | | 0 |
| 46 | HPMA | DEAA | | 90 | 10 | | | 0 |
| 47 | HPMA | DEAA | | 70 | 30 | | | 0 |
| 48 | HPMA | DEAA | | 50 | 50 | | | 0 |
| 49 | HPMA | DMAA | | 90 | 10 | | | 0 |
| 50 | HPMA | DMAA | | 70 | 30 | | | 0 |
| 51 | HPMA | DMAA | | 50 | 50 | | | 0 |
| 52 | HPMA | PAA | | 90 | 10 | | | 0 |
| 53 | HPMA | PAA | | 70 | 30 | | | 0 |
| 54 | HPMA | PAA | | 50 | 50 | | | 0 |
| 55 | HBMA | DEAA | | 90 | 10 | | | 1 |
| 56 | HBMA | DEAA | | 70 | 30 | | | 1 |
| 57 | HBMA | DEAA | | 50 | 50 | | | 0 |
| 58 | HBMA | DMAA | | 90 | 10 | | | 0 |
| 59 | HBMA | DMAA | | 70 | 30 | | | 0 |
| 61 | HBMA | PAA | | 90 | 10 | | | 0 |
| 62 | HBMA | PAA | | 70 | 30 | | | 1 |
| 63 | HBMA | PAA | | 50 | 50 | | | 0 |
| 97 | MEMA | DEAEMA | | 70 | 30 | | | 0 |
| 98 | MEMA | DEAEMA | | 50 | 50 | | | 3 |
| 99 | MEMA | DMAEMA | | 90 | 10 | | | 0 |
| 100 | MEMA | DMAEMA | | 70 | 30 | | | 0 |
| 101 | MEMA | DMAEMA | | 50 | 50 | | | 0 |
| 102 | MEMA | DEAEA | | 90 | 10 | | | 0 |
| 102 | MEMA | DEAEA | | 90 | 10 | | | 0 |
| 103 | MEMA | DEAEA | | 70 | 30 | | | 0 |
| 104 | MEMA | DEAEA | | 50 | 50 | | | 2 |
| 105 | MEMA | DMAEA | | 90 | 10 | | | 0 |
| 106 | MEMA | DMAEA | | 70 | 30 | | | 0 |
| 108 | MEMA | MTEMA | | 90 | 10 | | | 0 |
| 109 | MEMA | MTEMA | | 70 | 30 | | | 1 |
| 110 | MEMA | MTEMA | | 50 | 50 | | | 0 |
| 111 | MEMA | BAEMA | | 90 | 10 | | | 2 |
| 112 | MEMA | BAEMA | | 70 | 30 | | | 2 |
| 114 | MEMA | DMAPMAA | | 90 | 10 | | | 0 |
| 115 | MEMA | DMAPMAA | | 70 | 30 | | | 0 |
| 116 | MEMA | DMAPMAA | | 50 | 50 | | | 0 |
| 117 | MEMA | BACOEA | | 90 | 10 | | | 0 |
| 118 | MEMA | BACOEA | | 70 | 30 | | | 0 |
| 119 | MEMA | BACOEA | | 50 | 50 | | | 0 |
| 120 | MEMA | DMVBA | | 90 | 10 | | | 0 |
| 121 | MEMA | DMVBA | | 70 | 30 | | | 0 |
| 123 | MEMA | VAA | | 90 | 10 | | | 0 |
| 124 | MEMA | VAA | | 70 | 30 | | | 0 |
| 126 | MEMA | VI | | 90 | 10 | | | 0 |
| 127 | MEMA | VI | | 70 | 30 | | | 0 |
| 128 | MEMA | VI | | 50 | 50 | | | 1 |
| 129 | MEMA | VPNO | | 90 | 10 | | | 0 |
| 130 | MEMA | VPNO | | 70 | 30 | | | 0 |
| 131 | MEMA | VPNO | | 50 | 50 | | | 0 |
| 133 | MEMA | VP-4 | | 70 | 30 | | | 0 |
| 134 | MEMA | VP-4 | | 50 | 50 | | | 2 |
| 135 | MEMA | VP-2 | | 90 | 10 | | | 0 |
| 136 | MEMA | VP-2 | | 70 | 30 | | | 0 |
| 137 | MEMA | VP-2 | | 50 | 50 | | | 1 |
| 138 | MEMA | DAAA | | 90 | 10 | | | 0 |
| 139 | MEMA | DAAA | | 70 | 30 | | | 1 |
| 140 | MEMA | DAAA | | 50 | 50 | | | 0 |
| 141 | MEMA | MNPMA | | 90 | 10 | | | 0 |
| 142 | MEMA | MNPMA | | 70 | 30 | | | 0 |
| 143 | MEMA | MNPMA | | 50 | 50 | | | 1 |
| 150 | HEMA | DEAEMA | | 90 | 10 | | | 0 |
| 152 | HEMA | DEAEMA | | 50 | 50 | | | 1 |
| 153 | HEMA | DMAEMA | | 90 | 10 | | | 0 |
| 156 | HEMA | DEAEA | | 90 | 10 | | | 0 |
| 157 | HEMA | DEAEA | | 70 | 30 | | | 0 |
| 158 | HEMA | DEAEA | | 50 | 50 | | | 0 |
| 159 | HEMA | DMAEA | | 90 | 10 | | | 0 |
| 160 | HEMA | DMAEA | | 70 | 30 | | | 0 |
| 161 | HEMA | DMAEA | | 50 | 50 | | | 0 |
| 162 | HEMA | MTEMA | | 90 | 10 | | | 0 |
| 163 | HEMA | MTEMA | | 70 | 30 | | | 1 |
| 164 | HEMA | MTEMA | | 50 | 50 | | | 0 |
| 165 | HEMA | BAEMA | | 90 | 10 | | | 0 |
| 167 | HEMA | BAEMA | | 50 | 50 | | | 2 |
| 168 | HEMA | DMAPMAA | | 90 | 10 | | | 1 |
| 169 | HEMA | DMAPMAA | | 70 | 30 | | | 0 |
| 170 | HEMA | DMAPMAA | | 50 | 50 | | | 0 |
| 171 | HEMA | BACOEA | | 90 | 10 | | | 1 |
| 172 | HEMA | BACOEA | | 70 | 30 | | | 0 |
| 173 | HEMA | BACOEA | | 50 | 50 | | | 1 |
| 174 | HEMA | DMVBA | | 90 | 10 | | | 0 |
| 175 | HEMA | DMVBA | | 70 | 30 | | | 0 |
| 175 | HEMA | DMVBA | | 70 | 30 | | | 0 |
| 176 | HEMA | DMVBA | | 50 | 50 | | | 2 |
| 177 | HEMA | VAA | | 90 | 10 | | | 1 |
| 178 | HEMA | VAA | | 70 | 30 | | | 0 |
| 179 | HEMA | VAA | | 50 | 50 | | | 0 |
| 180 | HEMA | VI | | 90 | 10 | | | 1 |
| 181 | HEMA | VI | | 70 | 30 | | | 2 |
| 182 | HEMA | VI | | 50 | 50 | | | 0 |
| 184 | HEMA | VPNO | | 70 | 30 | | | 0 |
| 185 | HEMA | VPNO | | 50 | 50 | | | 1 |
| 186 | HEMA | VP-4 | | 90 | 10 | | | 0 |
| 187 | HEMA | VP-4 | | 70 | 30 | | | 2 |
| 189 | HEMA | VP-2 | | 90 | 10 | | | 1 |
| 190 | HEMA | VP-2 | | 70 | 30 | | | 1 |
| 192 | HEMA | DAAA | | 90 | 10 | | | 1 |
| 193 | HEMA | DAAA | | 70 | 30 | | | 0 |
| 194 | HEMA | DAAA | | 50 | 50 | | | 1 |
| 195 | HEMA | MNPMA | | 90 | 10 | | | 0 |
| 196 | HEMA | MNPMA | | 70 | 30 | | | 0 |
| 197 | HEMA | MNPMA | | 50 | 50 | | | 0 |
| 199 | MMA | A-H | | 70 | 30 | | | 0 |
| 200 | MMA | A-H | | 50 | 50 | | | 0 |
| 201 | MMA | AES-H | | 90 | 10 | | | 0 |
| 202 | MMA | AES-H | | 70 | 30 | | | 1 |
| 203 | MMA | AES-H | | 50 | 50 | | | 0 |
| 205 | MMA | MA-H | | 70 | 30 | | | 0 |
| 206 | MMA | MA-H | | 50 | 50 | | | 0 |
| 207 | MMA | AAG-H | | 90 | 10 | | | 0 |
| 208 | MMA | AAG-H | | 70 | 30 | | | 0 |
| 209 | MMA | AAG-H | | 50 | 50 | | | 0 |
| 214 | MEMA | A-H | | 70 | 30 | | | 1 |
| 215 | MEMA | A-H | | 50 | 50 | | | 0 |
| 216 | MEMA | AES-H | | 90 | 10 | | | 1 |
| 218 | MEMA | AES-H | | 50 | 50 | | | 1 |
| 222 | MEMA | AAG-H | | 90 | 10 | | | 0 |
| 223 | MEMA | AAG-H | | 70 | 30 | | | 0 |
| 228 | MMA | A-H | DEAEMA | 70 | 20 | 10 | | 1 |
| 229 | MMA | A-H | DEAEMA | 70 | 15 | 15 | | 1 |
| 230 | MMA | A-H | DEAEMA | 70 | 10 | 20 | | 1 |
| 231 | MMA | A-H | DEAEA | 70 | 20 | 10 | | 0 |
| 232 | MMA | A-H | DEAEA | 70 | 15 | 15 | | 0 |
| 233 | MMA | A-H | DEAEA | 70 | 10 | 20 | | 0 |
| 234 | MMA | MA-H | DEAEMA | 70 | 20 | 10 | | 0 |
| 235 | MMA | MA-H | DEAEMA | 70 | 15 | 15 | | 0 |
| 236 | MMA | MA-H | DEAEMA | 70 | 10 | 20 | | 0 |
| 237 | MMA | MA-H | DEAEA | 70 | 20 | 10 | | 0 |
| 238 | MMA | MA-H | DEAEA | 70 | 15 | 15 | | 0 |
| 240 | MEMA | A-H | DEAEMA | 70 | 20 | 10 | | 0 |
| 243 | MEMA | A-H | DEAEA | 70 | 20 | 10 | | 0 |
| 245 | MEMA | A-H | DEAEA | 70 | 10 | 20 | | 0 |
| 246 | MEMA | MA-H | DEAEMA | 70 | 20 | 10 | | 0 |
| 248 | MEMA | MA-H | DEAEMA | 70 | 10 | 20 | | 0 |
| 249 | MEMA | MA-H | DEAEA | 70 | 20 | 10 | | 0 |
| 251 | MEMA | MA-H | DEAEA | 70 | 10 | 20 | | 0 |
| 252 | MEMA | GMA | | 90 | 10 | | | 0 |
| 255 | MEMA | GMA | | 90 | 10 | | DnBA | 2 |
| 258 | MEMA | GMA | | 90 | 10 | | DnHA | 0 |
| 260 | MEMA | GMA | | 50 | 50 | | DnHA | 0 |
| 262 | MEMA | GMA | | 70 | 30 | | DcHA | 0 |
| 264 | MEMA | GMA | | 90 | 10 | | DBnA | 0 |
| 267 | MEMA | GMA | | 90 | 10 | | MnHA | 0 |
| 268 | MEMA | GMA | | 70 | 30 | | MnHA | 1 |
| 273 | MEMA | GMA | | 90 | 10 | | BnMA | 0 |
| 279 | MEMA | GMA | | 90 | 10 | | Pyre | 0 |
| 280 | MEMA | GMA | | 70 | 30 | | Pyre | 0 |
| 281 | MEMA | GMA | | 50 | 50 | | Pyre | 1 |
| 285 | MEMA | GMA | | 90 | 10 | | MAn | 2 |
| 291 | MEMA | GMA | | 90 | 10 | | DEMEDA | 0 |
| 294 | MEMA | GMA | | 90 | 10 | | TMPDA | 1 |
| 295 | MEMA | GMA | | 70 | 30 | | TMPDA | 2 |
| 296 | MEMA | GMA | | 50 | 50 | | TMPDA | 2 |
| 303 | MMA | GMA | | 90 | 10 | | | 1 |
| 304 | MMA | GMA | | 70 | 30 | | | 0 |
| 305 | MMA | GMA | | 50 | 50 | | | 1 |
| 306 | MMA | GMA | | 90 | 10 | | DnBA | 1 |
| 309 | MMA | GMA | | 90 | 10 | | DnHA | 3 |
| 316 | MMA | GMA | | 70 | 30 | | DBnA | 3 |
| 317 | MMA | GMA | | 50 | 50 | | DBnA | 3 |
| 318 | MMA | GMA | | 90 | 10 | | MnHA | 2 |
| 319 | MMA | GMA | | 70 | 30 | | MnHA | 2 |
| 321 | MMA | GMA | | 90 | 10 | | cHMA | 3 |
| 322 | MMA | GMA | | 70 | 30 | | cHMA | 1 |
| 323 | MMA | GMA | | 50 | 50 | | cHMA | 1 |
| 324 | MMA | GMA | | 90 | 10 | | BnMA | 2 |
| 325 | MMA | GMA | | 70 | 30 | | BnMA | 0 |
| 326 | MMA | GMA | | 50 | 50 | | BnMA | 2 |
| 327 | MMA | GMA | | 90 | 10 | | MAEPy | 0 |
| 329 | MMA | GMA | | 50 | 50 | | MAEPy | 2 |
| 330 | MMA | GMA | | 90 | 10 | | Pyrle | 0 |
| 331 | MMA | GMA | | 70 | 30 | | Pyrle | 0 |
| 332 | MMA | GMA | | 50 | 50 | | Pyrle | 0 |
| 336 | MMA | GMA | | 90 | 10 | | MAn | 0 |
| 338 | MMA | GMA | | 50 | 50 | | MAn | 3 |
| 339 | MMA | GMA | | 90 | 10 | | TMEDA | 0 |
| 341 | MMA | GMA | | 50 | 50 | | TMEDA | 0 |
| 342 | MMA | GMA | | 90 | 10 | | DEMEDA | 1 |
| 343 | MMA | GMA | | 70 | 30 | | DEMEDA | 0 |
| 345 | MMA | GMA | | 90 | 10 | | TMPDA | 0 |
| 348 | MMA | GMA | | 90 | 10 | | Mpi | 0 |
| 349 | MMA | GMA | | 70 | 30 | | Mpi | 0 |
| 353 | MMA | GMA | | 50 | 50 | | TEDETA | 0 |
| 354 | MMA | DEAEMA | | 90 | 10 | | | 2 |
| 355 | MMA | DEAEMA | | 70 | 30 | | | 1 |
| 356 | MMA | DEAEMA | | 50 | 50 | | | 1 |
| 357 | MMA | DMAEMA | | 90 | 10 | | | 1 |
| 359 | MMA | DMAEMA | | 50 | 50 | | | 0 |
| 360 | MMA | DEAEA | | 90 | 10 | | | 1 |
| 361 | MMA | DEAEA | | 70 | 30 | | | 1 |
| 363 | MMA | DMAEA | | 90 | 10 | | | 0 |
| 364 | MMA | DMAEA | | 70 | 30 | | | 2 |
| 365 | MMA | DMAEA | | 50 | 50 | | | 0 |
| 366 | HPMA | DEAEMA | | 90 | 10 | | | 0 |
| 367 | HPMA | DEAEMA | | 70 | 30 | | | 0 |
| 368 | HPMA | DEAEMA | | 50 | 50 | | | 0 |
| 369 | HPMA | DMAEMA | | 90 | 10 | | | 0 |
| 370 | HPMA | DMAEMA | | 70 | 30 | | | 0 |
| 371 | HPMA | DMAEMA | | 50 | 50 | | | 0 |
| 372 | HPMA | DEAEA | | 90 | 10 | | | 0 |
| 374 | HPMA | DEAEA | | 50 | 50 | | | 0 |
| 375 | HPMA | DMAEA | | 90 | 10 | | | 0 |
| 376 | HPMA | DMAEA | | 70 | 30 | | | 0 |
| 377 | HPMA | DMAEA | | 50 | 50 | | | 0 |
| 378 | HBMA | DEAEMA | | 90 | 10 | | | 1 |
| 380 | HBMA | DEAEMA | | 50 | 50 | | | 0 |
| 381 | HBMA | DMAEMA | | 90 | 10 | | | 1 |
| 382 | HBMA | DMAEMA | | 70 | 30 | | | 0 |
| 384 | HBMA | DEAEA | | 90 | 10 | | | 0 |
| 385 | HBMA | DEAEA | | 70 | 30 | | | 0 |
| 386 | HBMA | DEAEA | | 50 | 50 | | | 0 |
| 387 | HBMA | DMAEA | | 90 | 10 | | | 0 |
| 389 | HBMA | DMAEA | | 50 | 50 | | | 0 |
| 390 | EMA | DEAEMA | | 90 | 10 | | | 1 |
| 391 | EMA | DEAEMA | | 70 | 30 | | | 0 |
| 392 | EMA | DEAEMA | | 50 | 50 | | | 0 |
| 393 | EMA | DMAEMA | | 90 | 10 | | | 1 |
| 394 | EMA | DMAEMA | | 70 | 30 | | | 0 |
| 395 | EMA | DMAEMA | | 50 | 50 | | | 1 |
| 396 | EMA | DEAEA | | 90 | 10 | | | 1 |
| 397 | EMA | DEAEA | | 70 | 30 | | | 0 |
| 398 | EMA | DEAEA | | 50 | 50 | | | 0 |
| 399 | EMA | DMAEA | | 90 | 10 | | | 1 |
| 400 | EMA | DMAEA | | 70 | 30 | | | 1 |
| 401 | EMA | DMAEA | | 50 | 50 | | | 1 |
| 402 | BMA | DEAEMA | | 90 | 10 | | | 0 |
| 403 | BMA | DEAEMA | | 70 | 30 | | | 0 |
| 404 | BMA | DEAEMA | | 50 | 50 | | | 0 |
| 405 | BMA | DMAEMA | | 90 | 10 | | | 0 |
| 406 | BMA | DMAEMA | | 70 | 30 | | | 1 |
| 407 | BMA | DMAEMA | | 50 | 50 | | | 0 |
| 408 | BMA | DEAEA | | 90 | 10 | | | 1 |
| 410 | BMA | DEAEA | | 50 | 50 | | | 0 |
| 411 | BMA | DMAEA | | 90 | 10 | | | 1 |
| 412 | BMA | DMAEA | | 70 | 30 | | | 0 |
| 413 | BMA | DMAEA | | 50 | 50 | | | 0 |
| 414 | MEMA | DEAEMA | MA | 40 | 30 | 30 | | 1 |
| 415 | MEMA | DEAEMA | MA | 60 | 10 | 30 | | 0 |
| 416 | MEMA | DEAEMA | MA | 60 | 30 | 10 | | 0 |
| 417 | MEMA | DEAEMA | MA | 80 | 10 | 10 | | 0 |
| 418 | MEMA | DEAEA | MA | 40 | 30 | 30 | | 0 |
| 419 | MEMA | DEAEA | MA | 60 | 10 | 30 | | 1 |
| 420 | MEMA | DEAEA | MA | 60 | 30 | 10 | | 0 |
| 421 | MEMA | DEAEA | MA | 80 | 10 | 10 | | 0 |
| 422 | MEMA | DEAEMA | BMA | 40 | 30 | 30 | | 0 |
| 423 | MEMA | DEAEMA | BMA | 60 | 10 | 30 | | 0 |
| 424 | MEMA | DEAEMA | BMA | 60 | 30 | 10 | | 1 |
| 425 | MEMA | DEAEMA | BMA | 80 | 10 | 10 | | 0 |
| 426 | MEMA | DEAEA | BMA | 40 | 30 | 30 | | 3 |
| 428 | MEMA | DEAEA | BMA | 60 | 30 | 10 | | 0 |
| 429 | MEMA | DEAEA | BMA | 80 | 10 | 10 | | 1 |
| 430 | MEMA | DEAEMA | MEA | 40 | 30 | 30 | | 0 |
| 431 | MEMA | DEAEMA | MEA | 60 | 10 | 30 | | 0 |
| 432 | MEMA | DEAEMA | MEA | 60 | 30 | 10 | | 1 |
| 433 | MEMA | DEAEMA | MEA | 80 | 10 | 10 | | 1 |
| 434 | MEMA | DEAEA | MEA | 40 | 30 | 30 | | 0 |
| 435 | MEMA | DEAEA | MEA | 60 | 10 | 30 | | 0 |
| 436 | MEMA | DEAEA | MEA | 60 | 30 | 10 | | 1 |
| 437 | MEMA | DEAEA | MEA | 80 | 10 | 10 | | 0 |
| 438 | MEMA | DEAEMA | DEGMEMA | 40 | 30 | 30 | | 3 |
| 443 | MEMA | DEAEA | DEGMEMA | 60 | 10 | 30 | | 0 |
| 444 | MEMA | DEAEA | DEGMEMA | 60 | 30 | 10 | | 1 |
| 448 | MEMA | DEAEMA | THFFA | 60 | 30 | 10 | | 1 |
| 450 | MEMA | DEAEA | THFFA | 40 | 30 | 30 | | 0 |
| 452 | MEMA | DEAEA | THFFA | 60 | 30 | 10 | | 0 |
| 453 | MEMA | DEAEA | THFFA | 80 | 10 | 10 | | 0 |
| 458 | MEMA | DEAEA | THFFMA | 40 | 30 | 30 | | 0 |
| 459 | MEMA | DEAEA | THFFMA | 60 | 10 | 30 | | 1 |
| 460 | MEMA | DEAEA | THFFMA | 60 | 30 | 10 | | 0 |
| 465 | MEMA | DEAEMA | HEA | 80 | 10 | 10 | | 0 |
| 467 | MEMA | DEAEA | HEA | 60 | 10 | 30 | | 0 |
| 468 | MEMA | DEAEA | HEA | 60 | 30 | 10 | | 0 |
| 469 | MEMA | DEAEA | HEA | 80 | 10 | 10 | | 0 |
| 470 | MEMA | DEAEMA | HEMA | 40 | 30 | 30 | | 0 |
| 474 | MEMA | DEAEA | HEMA | 40 | 30 | 30 | | 0 |
| 475 | MEMA | DEAEA | HEMA | 60 | 10 | 30 | | 0 |
| 476 | MEMA | DEAEA | HEMA | 60 | 30 | 10 | | 0 |
| 477 | MEMA | DEAEA | HEMA | 80 | 10 | 10 | | 1 |
| 481 | MEMA | DEAEMA | A-H | 80 | 10 | 10 | | 1 |
| 485 | MEMA | DEAEA | A-H | 80 | 10 | 10 | | 0 |
| 493 | MEMA | DEAEA | MA-H | 80 | 10 | 10 | | 0 |
| 496 | MEMA | DEAEMA | DMAA | 60 | 30 | 10 | | 0 |
| 497 | MEMA | DEAEMA | DMAA | 80 | 10 | 10 | | 0 |
| 500 | MEMA | DEAEA | DMAA | 60 | 30 | 10 | | 0 |
| 501 | MEMA | DEAEA | DMAA | 80 | 10 | 10 | | 0 |
| 502 | MEMA | DEAEMA | DAAA | 40 | 30 | 30 | | 1 |
| 503 | MEMA | DEAEMA | DAAA | 60 | 10 | 30 | | 0 |
| 506 | MEMA | DEAEA | DAAA | 40 | 30 | 30 | | 2 |
| 507 | MEMA | DEAEA | DAAA | 60 | 10 | 30 | | 1 |
| 508 | MEMA | DEAEA | DAAA | 60 | 30 | 10 | | 0 |
| 509 | MEMA | DEAEA | DAAA | 80 | 10 | 10 | | 1 |
| 511 | MEMA | DEAEMA | MMA | 60 | 10 | 30 | | 0 |
| 512 | MEMA | DEAEMA | MMA | 60 | 30 | 10 | | 2 |
| 513 | MEMA | DEAEMA | MMA | 80 | 10 | 10 | | 1 |
| 514 | MEMA | DEAEA | MMA | 40 | 30 | 30 | | 1 |
| 515 | MEMA | DEAEA | MMA | 60 | 10 | 30 | | 0 |
| 516 | MEMA | DEAEA | MMA | 60 | 30 | 10 | | 2 |
| 517 | MEMA | DEAEA | MMA | 80 | 10 | 10 | | 1 |
| 518 | MEMA | DEAEMA | St | 40 | 30 | 30 | | 0 |
| 519 | MEMA | DEAEMA | St | 60 | 10 | 30 | | 1 |
| 520 | MEMA | DEAEMA | St | 60 | 30 | 10 | | 1 |
| 522 | MEMA | DEAEA | St | 40 | 30 | 30 | | 1 |
| 523 | MEMA | DEAEA | St | 60 | 10 | 30 | | 0 |
| 525 | MEMA | DEAEMA | | 85 | 15 | | | 0 |
| 526 | MEMA | DEAEMA | | 80 | 20 | | | 0 |
| 527 | MEMA | DEAEMA | | 75 | 25 | | | 0 |
| 528 | MEMA | DEAEMA | | 70 | 30 | | | 0 |
| 529 | MEMA | DEAEMA | | 65 | 35 | | | 1 |
| 530 | MEMA | DEAEMA | | 60 | 40 | | | 0 |
| 531 | MEMA | DEAEMA | | 55 | 45 | | | 2 |
| 532 | MEMA | A-H | DEAEMA | 85 | 5 | 10 | | 0 |
| 533 | MEMA | A-H | DEAEMA | 80 | 5 | 15 | | 0 |
| 534 | MEMA | A-H | DEAEMA | 75 | 5 | 20 | | 0 |
| 535 | MEMA | A-H | DEAEMA | 70 | 5 | 25 | | 0 |
| 536 | MEMA | A-H | DEAEMA | 65 | 5 | 30 | | 0 |
| 537 | MEMA | A-H | DEAEMA | 60 | 5 | 35 | | 0 |
| 538 | MEMA | A-H | DEAEMA | 55 | 5 | 40 | | 0 |
| 539 | MEMA | A-H | DEAEMA | 50 | 5 | 45 | | 0 |
| 540 | MEMA | A-H | DEAEMA | 75 | 10 | 15 | | 0 |
| 541 | MEMA | A-H | DEAEMA | 70 | 10 | 20 | | 0 |
| 542 | MEMA | A-H | DEAEMA | 65 | 10 | 25 | | 1 |
| 543 | MEMA | A-H | DEAEMA | 55 | 10 | 35 | | 0 |
| 544 | MEMA | A-H | DEAEMA | 50 | 10 | 40 | | 0 |
| 545 | MEMA | A-H | DEAEMA | 65 | 15 | 20 | | 0 |
| 546 | MEMA | A-H | DEAEMA | 60 | 15 | 25 | | 0 |
| 547 | MEMA | A-H | DEAEMA | 55 | 15 | 30 | | 0 |
| 548 | MEMA | A-H | DEAEMA | 50 | 15 | 35 | | 0 |
| 549 | MEMA | A-H | DEAEMA | 55 | 20 | 25 | | 0 |
| 550 | MEMA | A-H | DEAEMA | 50 | 20 | 30 | | 0 |
| 551 | MEMA | A-H | DEAEMA | 90 | 5 | 5 | | 0 |
| 552 | MEMA | A-H | DEAEMA | 80 | 15 | 5 | | 0 |
| 553 | MEMA | A-H | DEAEMA | 70 | 25 | 5 | | 0 |
| 554 | MEMA | A-H | DEAEMA | 60 | 35 | 5 | | 0 |
| 555 | MEMA | A-H | DEAEMA | 50 | 45 | 5 | | 0 |
| 556 | MEMA | A-H | DEAEMA | 50 | 40 | 10 | | 0 |
| 557 | MEMA | A-H | DEAEMA | 60 | 25 | 15 | | 0 |
| 558 | MEMA | A-H | DEAEMA | 50 | 35 | 15 | | 0 |
| 559 | MEMA | A-H | DEAEMA | 60 | 20 | 20 | | 0 |
| 560 | MEMA | A-H | DEAEMA | 50 | 30 | 20 | | 0 |
| 561 | MEMA | A-H | DEAEMA | 50 | 25 | 25 | | 0 |
| 562 | St | GMA | | 90 | 10 | | | 0 |
| 563 | St | GMA | | 70 | 30 | | | 0 |
| 564 | HBMA | VP-2 | | 50 | 50 | | | 0 |

TABLE II
Nomenclature of the monomers

| Monomer | |
|---|---|
| Nomenclature | Name |
| AAG-H | 2-acrylamidoglycolic acid |
| AES-H | mono-2-(acryloyoxy)ethyl succinate |
| A-H | acrylic acid |
| BAEMA | 2-(tert-butylamino)ethyl methacrylate |
| BACOEA | 2-[[(butylamino)carbonyl]oxy]ethyl acrylate |
| BMA | n-butyl methacrylate |
| DAAA | diacetone acrylamide |
| DEAA | diethylacrylamide |
| DEAEA | 2-(diethylamino)ethyl acrylate |
| DEAEMA | diethylaminoethyl methacrylate |
| DEGMEMA | di(ethyleneglycol) methyl ether methacrylate |
| DMAA | dimethyl acrylamide |
| DMAPMAA | N-[3-(dimethylamino)propyl] methacrylamide |
| DMVBA | N,N-dimethylvinylbenzylamine |
| EMA | ethyl methacrylate |
| GMA | glycidyl methacrylate |
| HBMA | hydroxybutyl methacrylate |
| HEA | 2-hydroxyethyl acrylate |
| HEMA | 2-hydroxyethyl methacrylate |
| HPMA | hydroxypropylmethacrylate |
| MA | methyl Acrylate |
| MA-H | methacrylic acid |
| MEA | 2-methoxyethyl acrylate |
| MMA | methyl methacrylate |
| MEMA | methoxyethyl methacrylate |
| MTEMA | 2-(methylthio)ethyl methacrylate |
| MNPMA | 2-methyl-2-nitropropyl methacrylate |
| PAA | N-isopropyl acrylamide |
| St | styrene |
| THFFA | tetrahydrofurfuryl acrylate |
| THFFMA | tetrahydrofurfuryl methacrylate |
| VAA | N-vinylacetamide |
| VI | 1-vinylimidazol |
| VP-2 | 2-vinylpyridine |
| VP-4 | 4-vinylpyridine |
| VPNO | 1-vinyl-2-pyrrolidinone |

TABLE III
Nomenclature of the amines use
in the derivatized polymers

| Amine | |
|---|---|
| Nomenclature | Name |
| BnMA | N-benzylmethylamine |
| cHMA | cyclohexanemethylamine |
| DBnA | dibenzylamine |
| DcHA | dicyclohexylamine |
| DnHA | di-n-hexylamine |
| DEMEDA | N,N-diethyl-N′-methylethylenediamine |
| DnBA | di-n-butylamine |
| MAEPy | 2-(2-methylaminoethyl)pyridine |
| MAn | N-methylailine |
| MnHA | N-methylhexylamine |
| Mpi | 1-methylpiperazine |
| Pyrle | pyrrole |
| TEDETA | N,N,N′,N′-tetraethyldiethylenetriamine |
| TMEDA | N,N,N-trimethylethylenediamine |
| TMPDA | N,N,N′-trimethyl-1,3-propanediamine |

Solutions (1% w/v) of the polymers in N-methylpyrrolidone (NMP) were placed into microwell plates and then printed on agarose-coated slides using a contact printer (QArraymini, Genetix, UK) with 32 aQu solid pins (K2785, Genetix). The printing conditions were 5 stamps per spot, with a 100 sec⁻³ inking timing and a 200 sec⁻³ stamping time. The printed slides were dried in a vacuum oven (45° C.) overnight to remove the remaining NMP. Polymer microarrays were sterilized for 30 min under UV light before using for cell culture.

### Human-Derived Aortic Valve Interstitial Cells Isolation and Culture

Primary human aortic valve interstitial cells VICs were isolated by enzymatic dissociation of surgically removed AoVs at the time of after surgical valve replacement. Samples were collected for research use, after approval by the Local Ethical committee, and upon informed consent of the patient. Briefly, the isolation protocol, as previously described in [22], started with the incubation of the healthy (non-calcific) portions of the leaflets for 5 minutes on shaker at 37° C. in Collagenase Type II solution (1000 U/ml, Worthington), to remove the endothelial layer. A second incubation for 2 hrs under the same conditions served for aVICs isolation. Cells were plated for ex-vivo amplification on a 1% gelatin coated plastic cell culture dishes (10 cm diameter), and cultured in a “complete medium”, made of DMEM (Lonza) supplemented with 150 U/ml penicillin/streptomycin (Sigma Aldrich), 2 mM L-glutamine (Sigma Aldrich) and 10% bovine serum (HyClone, Thermo Scientific). Cells were expanded for up to four passages before being employed for experiments.

### Polymers Microarray Screening

Following expansion, aortic VICs isolated from 3 independent donors were seeded (3×10⁵ cells/array) and cultured for 72 h onto PAs microarrays in duplicate. The arrays were housed in an purpose-made manufactured polycarbonate chamber, designed to circumscribe an area around the array, optimizing the seeding efficiency and minimizing the volume of media. At the end of the culture period, arrays were fixed in 4% paraformaldehyde (4% PFA) for 20 minutes, washed in phosphate buffered saline (PBS) and stained for 4′,6-diamidin-2-fenilindole (DAPI), phalloidin, vimentin, collagen type I and alpha smooth muscle actin (αSMA). Immunofluorescence images were acquired using a Nikon Eclipse TE200 or a Zeiss Apotome fluorescence microscope (Carl Zeiss, Jena, Germany), through z-stack reconstruction. The adhesion of VICs on the different PAs after 72 hours of culture was evaluated using automated counting of the number of nuclei per spot: cell nuclei stained with DAPI were quantified by implementation of the Analyze Particles tool of ImageJ software (National Institute of Health, Bethesda, Md.). In order to derive a priority list of the materials to be implemented in a secondary screening, criteria to obtain a ranking of the PA success to induce cell adhesion were established. PAs were then classified by assigning a score=3 to PAs that promoted adhesion of the cells from all donors on at least 3 out of 4 materials replica spots averaged on all tested arrays; a score=2 to PAs promoting adhesion on at least 2 out of 4 materials replica spots; a score=1 to PAs promoting adhesion on at least 1 out of 4 materials replica spots, and a score=0 to all the others.

### Scale-Up and Validation of ‘Hit’ Polymers

Seven out of the nine ‘hit’ polymers identified from microarray primary screening according to the criteria described above, were synthesized by free-radical polymerization and characterized by gel permeation chromatography (GPC) and infrared spectroscopy (IR). GPC was conducted on an Agilent 1100 instrument, fitted with a PLGel 5 μm MIXED-C column (300×7.5 mm), with NMP as the eluent (flow rate 1 mL min⁻¹). The GPC was pre-calibrated using polystyrene standards. IR analysis was conducted using a Brucker Tensor 27 spectrometer.

Polymers were spin-coated onto circular glass coverslips. Two sizes of cover slips were used, Ø 19 mm and Ø 32 mm, respectively dedicated to immunofluorescence and gene expression analysis. Polymer solutions in THF (2% w/v) were spin-coated at 2000 rpm for 10 seconds using a desktop spin coater (6708D, Speedline technologies). The coated coverslips were dried in a convection oven at 40° C. overnight and sterilized using UV light prior to using for cell culture and housed in either 6- or 12-well plates previously coated with agarose (1% w/v). Coated coverslips, before use, were sterilised with UV light for 30 min.

aVICs Culture onto 2-D Scale-Up Coated Coverslips

aVICs were seeded onto coated coverslips at a cell density of 2000 cell/mm². Following 7 days of culture, immunofluorescence analysis (3 independent cell donors) were performed including staining for Phalloidin, Collagen type I, αSMA and DAPI. Images were acquired by confocal microscopy (LSM 710; Carl Zeiss, Jena, Germany). Automated cell counting (ImageJ) of nuclei per frame (A=0.7 mm²) was performed, averaging the results of 3 frames per sample.

RNA was extracted from cells cultured on the scale-up system for 7 and 14 days, with Tripure reagent (Roche Diagnostics). Quantitative real-time PCR (qRT-PCR) amplifications were performed for GAPDH, BMP2, OPN, ALP, RUNX2 (primers details in Table2), using Power SYBR Green PCR Master Mix (Applied Biosystems) on a 7900 Fast Real-Time PCR System (Applied Biosystems). Gene expression levels are expressed in fold increase referred to housekeeping gene (GAPDH) at seeding.

TABLE IV
PCR Primers

| | | | SEQ |
|---|---|---|---|
| | | | ID |
| Gene | Direction | Sequence | NOs: |
| hALP | Forward | TCACTCTCCGAGATGGTGGT | 1 |
| hALP | Reverse | GTGCCCGTGGTCAATTCT | 2 |
| hRunx2 | Forward | TCTGGCCTTCCACTCTCAGT | 3 |
| hRunx2 | Reverse | GACTGGCGGGGTGTAAGTAA | 4 |
| hOPN | Forward | GAGGGCTTGGTTGTCAGC | 5 |
| hOPN | Reverse | CAATTCTCATGGTAGTGAGTTTTCC | 6 |
| hBMP2 | Forward | TGTATCGCAGGCACTCAGGTC | 7 |
| hBMP2 | Reverse | TTCCCACTCGTTTCTGGTAGTTCTT | 8 |
| hCOL | Forward | GGA CAC AGA GGT TTC AGT GG | 9 |
| I | | | |
| hCOL | Reverse | CCA GTA GCA CCA TCA TTT CC | 10 |
| I | | | |
| hCOL | Forward | AGC TAC GGC AAT CCT GAA CT | 11 |
| III | | | |
| hCOL | Reverse | GGG CCT TCT TTA CAT TTC CA | 12 |
| III | | | |
| hACTA2 | Forward | AGA GTT ACG AGT TGC CTG ATG | 13 |
| hACTA2 | Reverse | CTG TTG TAG GTG GTT TCA TGG A | 14 |
| hVCAN | Forward | AAC TTC CTA CGT ATG CAC CTG | 15 |
| hVCAN | Reverse | AAG TGG CTC CAT TAC GAC AG | 16 |

Transfer in 3D—Scaffolds Coating with PA98

A Polycaprolactone (PCL) scaffold (Mimetix® Electrospinning Company, Cambridge, UK) with a 2 mm thickness, a 2 cm diameter and ˜100 μm pore size, was used for 3D experiments, either as supplied (uncoated) or after coating with PA98. After removing the backing paper, coating was performed by dipping the scaffolds in a solution of PA G dissolved in acetone and air dried into a polypropylene 48-well plates in a fume hood. Coating time and concentration of polymer solution were experimentally set to respectively circa 1 sec and 1% w/v, after evaluating scaffold integrity and polymer loading by scanning electron microscopy (SEM) and IR spectroscopy. SEM was conducted using a Hitachi 4700 II cold Field-emission Scanning Electron Microscope while IR analysis was conducted using a Brucker Tensor 27 spectrometer. Scaffolds, uncoated or coated under these optimized conditions, were sterilized by 72 hours incubation in BASE128 (AL.CHI.MIA s.r.l.), an EC certified decontamination solution containing an antibiotic/antifungal mixture (Gentamicin, Vancomycin, Cefotaxime and Amphotericin B) and approved for employment in Tissue Banking.

### Transfer in 3D—Bioreactor-Assisted VICs Seeding and Culture

8 mm diameter cylinders of uncoated and coated scaffolds were seeded (9×10³ cells/scaffold) and cultured statically or dynamically for 1, 7 and 14 days, with aVICs isolated from 5 independent donors. For static seeding, scaffolds were housed in agarose-coated multiwells and a small volume of cell suspension (50 μl/scaffold, 1.5×10⁵ cells/scaffold) was slowly dispersed over the top surface. Cells were allowed to adhere to the scaffolds for 2 hours, before gently adding 2 ml of medium to cover the scaffold. Dynamic culture was performed using the U-CUP bioreactor (Cellec Biotek AG, Basel, CH), a previously described direct perfusion system [23]. In our experiment VICs (4.5×10⁵ cells/scaffold) suspended in 9 ml complete medium were perfusion-seeded into the scaffolds at a 3 ml/min flow rate for 16 hours [24]. Thereafter, scaffolds were either harvested (day 1 experimental time point) or, following complete medium renewal, further cultured under perfusion at a 0.3 ml/min flow rate for 7 or 14 days. Medium change was performed twice per week. At harvest, replicas of both static and perfused samples were rinsed in PBS and cut into two halves, in order to proceed with different tests.

For RNA extraction, cellularized scaffolds were incubated in 500 μl Trizol reagent and RNA was isolated using the Direct-Zol RNA kit (Zhymo Research). Quantitative real-time PCR (qRT-PCR) amplifications were performed for GAPDH, COLI, COLIII, BMP2, OPN, ALP, RUNX2, ACTA2, VCAN (primers details in Table 1), using Power SYBR Green PCR Master Mix (Applied Biosystems) on a 7900 Fast Real-Time PCR System (Applied Biosystems). Gene expression levels are expressed in fold increase referred to housekeeping gene (GAPDH) at seeding.

Incubation of specimens for 3 h with 0.12 mM MTT (3(4,5-dimethylthiazol-2-yl)-2,5-diphenyltetrazolium bromide; Producer) was performed to qualitatively highlight cell distribution onto the scaffold. To perform histology and immunofluorescence analysis, after overnight fixation at 4° C. in PFA (4%), samples were incubated overnight at 4° C. in sucrose (15%), and, finally, included in a solution of sucrose (15%) and bovine skin gelatin (7.5%, Sigma Aldrich). Transversal cross-sections (10 μm thickness), obtained by cryo-sectioning, were stained with DAPI, Phalloidin and αSMA/Collagen I antibodies. Cell nuclei quantification was obtained by analyzing 3 transversal sections per each donor and condition (acquiring the whole section length).

### Sample Preparation for Mass Spectrometry

Coated and uncoated scaffolds used for the culture of VICs from 3 different donors were cut in small pieces 1 mm2 and subsequently washed 3 times with PBS. In order to decellularize the scaffolds, the samples were incubated with 500 μl of 0.25% v/v Triton X-100 (Sigma Aldrich) at 37° C. for 15 minutes with gentle agitation. After removal of the supernatant, containing cells, scaffold samples were vigorously washed 7 times with ice cold water to completely eliminate Triton X-100. 100 μl of 25 mmol/L NH4HCO3 containing 0.1% w/v RapiGest SF were then added for tryptic digestion. Samples were reduced with 5 mmol/L TCEP (tris(2-carboxyethyl)phosphine), dissolved in 100 mmol/L NH4HCO3, at room temperature for 30 min, and then carbamidomethylated with 10 mmol/L iodacetamide for 30 min at room temperature. Digestion was performed overnight at 37° C. using 0.5 μg of sequencing grade trypsin (Promega, Milan, Italy). After digestion, 2% v/v TFA was added to hydrolyse RapiGest SF and inactivate trypsin, and the solution was incubated at 37° C. for 40 min before being vortexed and centrifuged at 13,000 g for 10 minutes to eliminate RapiGest SF.

### Label-Free LC-MSE Analysis

Tryptic digests from coated and uncoated samples were then prepared adding yeast alcohol dehydrogenase (ADH) digest and Hi3 E. coli standards (Waters Corporation, Milford, Mass., USA) at the final concentration of 12.5 fmol/μl, as internal standards for molar amount estimation (Silva 2006) and quality controls.

Tryptic peptides separation was conducted with a TRIZAIC nanoTile (Waters Corporation, Milford, Mass., USA) using a nano-ACQUITY-UPLC System coupled to a SYNAPT-MS Mass Spectrometer equipped with a TRIZAIC source (Waters Corporation, Milford, Mass., USA). The TRIZAIC nanoTile used for this study, Acquity HSS T3, integrates a trapping column (5 μm, 180 μm×20 mm) for desalting and an analytical column (1.8 μm, 85 μm×100 mm) for peptide separation with an high level of reproducibility of retention time. Elution was performed at a flow rate of 550 nL/min by increasing the concentration of solvent B (0.1% formic acid in acetonitrile) from 3 to 40% in 90 min, using 0.1% formic acid in water as reversed phase solvent A[25]. 4 μl of tryptic digest were analysed in triplicate for each biological sample. Calibration and lockmass correction were performed as previously described[26]. Precursor ion masses and their fragmentation spectra were acquired in MSE mode as previously described[26] in order to obtain a qualitative and quantitative analysis of proteins associated with coated and uncoated scaffolds.

The software Progenesis QI for proteomics (Version 2.0, Nonlinear Dynamics, Newcastle upon Tyne, UK) was used for the quantitative analysis of peptide features and protein identification. Analysis of the data by Progenesis QI included retention time alignment to a reference sample selected by the software, feature filtering (based on retention time and charge (>2)), normalization considering all features, peptide search and multivariate statistical analysis. The principle of the search algorithm has been previously described in detail (Li 2009). The following criteria were used for protein identification:1 missed cleavage, Carbamidomethyl cysteine fixed and methionine oxidation as variable modifications. A UniProt database (release 2015-3; number of human sequence entries, 20199; number of bovin sequence entries, 6870) was used for database searches.

Fold changes in the quantitative expression, p-value and Q-value were calculated with the statistical package included in Progenesis QI for proteomics, using only peptides uniquely associated to the proteins to quantify proteins that were part of a group. A p-value<0.05 was considered significant. The significance of the regulation level was determined at a 20% fold change, but only proteins quantified with at least 2 peptides were considered. The entire data set of differentially expressed proteins was further filtered, after manual inspection of the results, by considering only the proteins with the same modulation in at least two out of three biological replicates. The data set was also subjected to unsupervised PCA analysis.

### Results

### Polymer Array Screening Revealed a Selected Number of Polymers Compatible for Human Valve Interstitial Cells (VICs) Culture

Primary array screening allowed simultaneous evaluation of the adhesion of VICs on the 384 polymer library spotted onto the array. The average number of cells adhered on each polymer was employed as a quantitative criteria to select polymers promoting aVICs adhesion. Based on automated cell counting (ImageJ) performed on the nuclear staining by DAPI seven polymers reproducibly supported VICs adhesion by all the donors (FIG. 2A). As shown in FIG. 2B, the number of cells ranged between 18±3 to 60±6 (mean±SE).

TABLE V
Nomenclature and chemical composition of the selected ‘hit’ polymers

| | | | | Ratio (mol) | | | |
|---|---|---|---|---|---|---|---|
| PA number | Monomer (1) | Monomer (2) | Monomer (3) | M (1) | M (2) | M (3) | Functionalization |
| PA98 | MEMA | DEAEMA | — | 50 | 50 | | None |
| PA309 | MMA | GMA | | 90 | 10 | | DnHA |
| PA316 | MMA | GMA | | 70 | 30 | — | DBnA |
| PA317 | MMA | GMA | | 50 | 50 | | DBnA |
| PA321 | MMA | GMA | | 90 | 10 | — | cHMA |
| PA338 | MMA | GMA | | 50 | 50 | | MAn |
| PA426 | MEMA | DEAEA | BMA | 40 | 30 | 30 | None |

### 2D Scale Up

7 ‘hit’ polymers identified in the primary screening were then tested in a scale up experiment onto a series of glass slides coated with each of the selected polymers. This confirmed that all the selected polymers supported human VICs adhesion. The number of nuclei per frame, again quantified by automatic counting of DAPI-stained nuclei, is reported in FIG. 3A, with a representative immunofluorescence to detect cytoskeleton polymerization, expression of smooth muscle actin-α (αSMA) and Collagen I in polymer adhered VICs. This latter analysis was performed adopting the same photo-multiplication setting in confocal imaging, thus allowing detection of variable levels of the two markers, which suggests different degree of VIC myofibroblast conversion onto the selected polymers.

In order to detect whether culture onto the different polymers affected the expression of crucial genes involved in VIC conversion into osteogenic cells, the expression of the genes encoding for the bone morphogenetic protein 2 (BMP2), Alkaline phosphatase (ALP), Osteopontin (OPN) and the transcription factor Runx2 (RUNX2), were assessed by real time RT-PCR (FIG. 3B) at 7 and 14 days of culture onto the PAs-coated glass slides. These results were compared to each other and with the expression levels of the genes in cells cultured in standard culture plates. The results clearly indicated polymers (PA338, PA309, PA316, PA98) onto which VICs underwent a transient upregulation of the tested genes at 7 days followed by return to steady levels at day 14, and polymers that maintained higher levels of the genes at both times.

These data demonstrate the feasibility of polymers employment as novel materials to manufacture ‘off-the-self’ tissue engineered heart valves by employing VICs.

Coating of a 3D PCL Scaffold with PA98

Based on the results of the immune-histochemistry and the Q-RT-PCR, PA98 was chosen as a reference material to perform functionalization of the 3D PCL scaffold and perform 3D culture of human VICs. Fast evaporating solvents, acetone and tetrahydrofuran (THF), were investigated for their ability to solubilize PA98. Although THF dissolved the scaffold immediately, acetone maintained the relative stability of the PCL material. Further tests were then conducted with acetone. This included dipping for decreasing amounts of time followed by weighing to assess the weight loss after overnight drying in a fume hood. Weight loss was determined to be 59.2%, 15.8% and 3.5% for 5, 2 and 1 minutes dipping, respectively (FIG. 4A). Due to the excessive weight loss suffered by the scaffolds, even with 1 minute dipping, further tests were conducted by dipping the scaffolds in acetone for either 10 sec or 5 sec and the average weight loss was determined to be 1.9% and 1.5% respectively, as shown in the table VI below (n=3).

TABLE VI
Average weight loss

| Dipping | Weight of | Weight | % weight |
|---|---|---|---|
| time | scaffold (gm) | loss (gm) | loss |
| 10 secs | 0.03262 | 0.00083 | 2.5 |
| 10 secs | 0.03592 | 0.00076 | 2.1 |
| 10 secs | 0.03367 | 0.00032 | 1.0 |
| 5 secs | 0.03259 | 0.00067 | 2.1 |
| 5 secs | 0.03370 | 0.00065 | 1.9 |
| 5 secs | 0.03325 | 0.00022 | 0.7 |

Integrity of fibres within the scaffold was tested by comparing SEM images of dried scaffolds treated with acetone (dipping time 5 sec) or untreated scaffolds. This confirmed Acetone as a suitable solvent for coating. Since the porous structure of the scaffold is crucial for penetration and uniform distribution of cells during seeding with the bioreactor, the influence of concentration of polymer solution was then studied to avoid clogging of the mesh. PCL scaffolds were finally dip-coated for 1 sec with PA98 dissolved in acetone at 0.1%, 0.5%, or 1% (w/v) concentration, and dried (see table VII and FIG. 4A)

TABLE VII
Scaffold preparation

| PA98 concentration | Weight of | Weight gain | % polymer |
|---|---|---|---|
| (w/v) | scaffold (gm) | (gm) | loading |
| 1.0% | 0.03411 | 0.00545 | 16.0% |
| 0.5% | 0.03205 | 0.00248 | 7.7% |
| 0.1% | 0.03182 | 0.00011 | 0.3% |

SEM revealed that, a gradient existed for the polymer loading within the scaffold for all the concentrations studied, with the bottom part of the scaffold containing more PA98 than the top part, even with a 1% polymer solution, the scaffolds retained their pores (FIG. 4B). After the SEM studies, in order to further reduce the dipping time during coating and minimize the possible PCL fibres damage, loading on the scaffolds was determined for a brief dipping time of approximately 1 sec. IR spectroscopy revealed that under these conditions, polymer loading was found to increase from 0.3% to 7.7% and 16%, respectively with 0.1%, 0.5% and 1% solutions respectively (FIG. 4A).

VICs Culture into the PA98 Coated 3D Scaffold

VICs were seeded into the PA98-coated scaffold either by static or dynamic seeding followed by culturing for a period up to 14 days. The efficiency of the two scaffold cellularization procedures was monitored by MTT staining of the scaffolds at 1, 7 and 14 days after the beginning of the culture (FIG. 5A). While static seeding only relied on the ability of VICs to invade the porous structure of the coated/uncoated PCL scaffolds, the application of a forced perfusion determined a more uniform distribution of the cells in depth into the 3D environment. This was evident from the higher MTT levels observed in the coated and uncoated scaffolds cellularized by the dynamic VICs seeding, particularly at day 7. Furthermore, although the untreated PCL was able to host VICs on its own, coating of the scaffold with PA98 increased VICs content at all times after seeding, as evaluated by nuclear counting in sections of cellularized scaffolds by forced perfusion (FIG. 5B). This confirms the indication of the coated PCL scaffolds to host VICs for artificial valve tissue engineering provided in previous studies [27, 28] and indicated an the increased colonization and uniformity of cellular distribution in the 3D environment coated with PA98.

A gene expression survey was performed to assess the expression of valve relevant genes in VICs seeded into the 3D scaffolds. This analysis included mRNAs encoding for the human αSMA gene and for extracellular matrix components produced by VICs in the valve tissue such as Collagen I/111 and Versican Glycosamino-Glycan (GAG). As shown in FIG. 5C, none of these genes was major changed by seeding VICs in the 3D environment and by PA98 scaffold functionalization.

To explore the ability of the PA98-coated scaffold to promote deposition of extracellular matrix components inventors therefore performed a mass-spectrometry-based high-throughput and high-resolution quantification of the proteins secreted by VICs after 14 days culture on PA98-coated versus uncoated PCL scaffolds. This analysis was performed with the aim at deciphering the ability of the selected PA to promote matrix maturation inside the scaffold. The proteins released by the cells into PA98-coated and uncoated PCL scaffolds were analyzed by means of a label-free MS-based proteomic approach, LC-MSE, which allows both a qualitative and quantitative comparative analysis between coated and uncoated samples. Data processing compared a total of 1503 peptides corresponding to 100 human proteins and revealed that 12 of them were more abundant in coated scaffolds samples whereas 12 were less abundant, discriminating the two samples in the three biological replicates (FIG. 6A). Tables VIII and IX show, respectively, the list of the proteins identified in the PA98-coated and uncoated scaffolds ordered for fold change and statistical significance. As shown, the mostly upregulated protein corresponded to microfibril-associated glycoprotein 4 (MFAP-4), a protein that has been recently reported to be involved in extracellular fibril organization and elastic fibers assembly[29]. Interestingly, the increase of this protein in the PA98-coated scaffold was the result of a post-translational process, as the level of the MFAP-4 mRNA was not significantly upregulated (FIG. 6B). Given the important role of this protein for elastin fibers extracellular assembly, it is tempting to speculate that post-translational processing of the protein is affected by contact with the polyacrylate thus helping the VIC-mediated mature elastin deposition in the extracellular space of the scaffold. This last result, also corroborated by the finding the polymer induced a generalized higher deposition of other essential elastin-interacting molecules such as Fibronectin, Fibulin-2 and Emilin[30] in the scaffolds (Table X), suggests that coating conventional scaffolds with the selected PAs or even manufacturing scaffolds directly with the selected PAs, may instruct cells to organize valve-competent ECM molecules, thus helping a final maturation of the bioartificial tissue.

TABLE VIII
Significantly upregulated proteins in PA98-coated vs C VIC-seeded scaffold.

| | | | | | | fold |
|---|---|---|---|---|---|---|
| | | Peptides | | | Max | change |
| | UniProt | used for | | Anova | fold | CV |
| Description | Accession | quantitation | Score | P-value | change | (%) |
| Microfibril-associated | P55083 | 3/3 | 32.8 | 1.1⁻¹⁶ | 15.36 | 10.0 |
| glycoprotein | | | | | | |
| Annexin A1 | P04083 | 13/14 | 116.0 | 2.19⁻¹⁰ | 1.48 | 6.0 |
| Cathepsin B | P07858 | 3/4 | 39.44 | 1.33⁻⁸ | 1.92 | 28.6 |
| Neutral alpha-glucosidase | Q14697 | 4/5 | 34.7 | 5.62⁻⁶ | 1.39 | 50.5 |
| AB | | | | | | |
| Actin-related protein 3 | P61158 | 2/2 | 12.4 | 3.82⁻⁶ | 1.93 | 34.2 |
| Extended synaptotagtnin-1 | Q9BSJ8 | 2/3 | 16.5 | 0.000106 | 3.39 | 86.1 |
| Pyruvate kinase PKM | P14618 | 20/25 | 211.3 | 0.000951 | 1.42 | 14.2 |
| Clathrin heavy chain 1 | Q00610 | 2/18 | 111.8 | 0.01708 | 1.22 | 47.9 |
| Guanine nucleotide-binding | P63244 | 5/5 | 31.9 | 0.004361 | 1.30 | 23.1 |
| protein subunit beta-2-like 1 | | | | | | |
| ATP-citrate synthase | P53396 | 2/2 | 12.6 | 0.005665 | 1.51 | 16.1 |
| Ubiquitin-like modifier- | P22314 | 3/7 | 43.55 | 0.007769 | 2.61 | 18.6 |
| activating enzyme 1 | | | | | | |
| Calreticulin | P27797 | 2/5 | 36.04 | 0.019216 | 1.65 | 61.6 |

TABLE IX
Significantly upregulated proteins in C vs PA98-coated VIC-seeded scaffold

| | | | | | | fold |
|---|---|---|---|---|---|---|
| | | Peptides | | | Max | change |
| | UniProt | used for | | Anova | fold | CV |
| Description | Accession | quantification | Score | P-value | change | (%) |
| Lamin-B1 | P20700 | 7/5 | 31.6 | 2.46⁻¹¹ | 2.25 | 10.9 |
| Ubiquitin-40S ribosomal | P62979 | 4/5 | 37.3 | 1.42⁻⁹ | 1.63 | 10.4 |
| protein S27a | | | | | | |
| Annexin A6 | P08133 | 2/3 | 20.9 | 3.81⁻⁷ | 2.89 | 49.1 |
| D-3-phosphoglycerate | O43175 | 5/5 | 25.4 | 1.7⁻⁷ | 1.43 | 16.0 |
| dehydrogenase | | | | | | |
| ADP-ribosylation factor 3 | P61204 | 4/9 | 63.1 | 0.000132 | 1.64 | 25.3 |
| Cell division control protein | P60953 | 2/2 | 13.0 | 0.000519 | 1.51 | 9.6 |
| 42 homolog | | | | | | |
| Histone H1.4 | P10412 | 2/2 | 13.4 | 0.00854 | 1.70 | 27.8 |
| Histone H3.1 | P68431 | 6/7 | 32.5 | 0.017908 | 2.30 | 54.4 |
| Coagulation factor V | P12259 | 3/13 | 101.8 | 0.030755 | 1.37 | 19.7 |
| L-lactate dehydrogenase A | P00338 | 2/4 | 26.3 | 0.042941 | 1.27 | 3.3 |
| chain | | | | | | |
| Histone H4 | P62805 | 10/10 | 73.6 | 0.047149 | 2.38 | 83.3 |
| Heterogeneous nuclear | P22626 | 4/4 | 25.4 | 0.048413 | 1.81 | 38.5 |
| ribonucleoproteins A2/B1 | | | | | | |

TABLE X
Expression of Extracellular Matrix related
Proteins in PA98-coated vs. C scaffolds.

| | Anova | Max fold |
|---|---|---|
| Description | P-value | change |
| ENTILIN-1 | 0.783877 | 1.14 |
| Collagen alpha-3(VI) chain | 0.517931 | 1.15 |
| Vitronectin | 0.187077 | 1.16 |
| Collagen alpha-2(VI) chain | 0.388854 | 1.20 |
| Collagen alpha-1(VI) chain | 0.501731 | 1.33 |
| Fibronectin | 0.159572 | 1.96 |
| Microfibril-associated glycoprotein-4 | 1.10⁻¹⁶ | 15.36 |

### REFERENCES

1. David, T. E., Surgical treatment of aortic valve disease. Nat Rev Cardiol, 2013. 10(7): p. 375-86.
2. Kheradvar, A., et al., Emerging Trends in Heart Valve Engineering: Part II. Novel and Standard Technologies for Aortic Valve Replacement. Annals of Biomedical Engineering, 2014: p. 1-14.
3. Forcillo, J., et al., Carpentier-Edwards Pericardial Valve in the Aortic Position: 25-Years Experience. Ann Thorac Surg, 2013. 96(2): p. 486-493.
4. Grabenwoger, M., et al., Impact of glutaraldehyde on calcification of pericardial bioprosthetic heart valve material. Ann Thorac Surg, 1996. 62(3): p. 772-7.
5. Siddiqui, R. F., J. R. Abraham, and J. Butany, Bioprosthetic heart valves: modes of failure. Histopathology, 2009. 55(2): p. 135-144.
6. Z. Konakci, K., et al., Alpha-Gal on bioprostheses: xenograft immune response in cardiac surgery. European Journal of Clinical Investigation, 2005. 35(1): p. 17-23.
7. Naso, F., et al., First quantification of alpha-Gal epitope in current glutaraldehyde fixed heart valve bioprostheses. Xenotransplantation, 2013. 20(4): p. 252-261.
8. Naso, F., et al., Alpha-Gal detectors in xenotransplantation research: a word of caution. Xenotransplantation, 2012. 19(4): p. 215-220.
9. Galili, U., The [alpha]-gal epitope and the anti-Gal antibody in xenotransplantation and in cancer immunotherapy. Immunol Cell Biol, 2005. 83(6): p. 674-686.
10. Hülsmann, J., et al., Transplantation material bovine pericardium: biomechanical and immunogenic characteristics after decellularization vs. glutaraldehyde-fixing. Xenotransplantation, 2012. 19(5): p. 286-297.
11. Breuer, C. K., et al., Application of tissue-engineering principles toward the development of a semilunar heart valve substitute. Tissue Eng, 2004. 10(11-12): p. 1725-36.
12. Balguid, A., et al., Stress related collagen ultrastructure in human aortic valves-implications for tissue engineering. Journal of Biomechanics, 2008. 41(12): p. 2612-2617.
13. Balguid, A., et al., The role of collagen cross-links in biomechanical behavior of human aortic heart valve leaflets—relevance for tissue engineering. Tissue Eng, 2007. 13(7): p. 1501-11.
14. Hammer, P. E., et al., Straightening of curved pattern of collagen fibers under load controls aortic valve shape. Journal of Biomechanics, 2014. 47(2): p. 341-346.
15. Sacks, M. S., F. J. Schoen, and J. E. Mayer, Bioengineering Challenges for Heart Valve Tissue Engineering. Annual Review of Biomedical Engineering, 2009. 11(1): p. 289-313.
16. MacGrogan, D., et al., How to Make a Heart Valve: From Embryonic Development to Bioengineering of Living Valve Substitutes. Cold Spring Harbor Perspectives in Medicine, 2014. 4(11).
17. Ghanbari, H., et al., Polymeric heart valves: new materials, emerging hopes. Trends in Biotechnology, 2009. 27(6): p. 359-367.
18. Fallahiarezoudar, E., et al., A review of: Application of synthetic scaffold in tissue engineering heart valves. Materials Science and Engineering: C, 2015. 48(0): p. 556-565.
19. Kheradvar, A., et al., Emerging Trends in Heart Valve Engineering: Part I. Solutions for Future. Annals of Biomedical Engineering, 2014: p. 1-11.
20. van Loosdregt, I. A. E. W., et al., Cell-mediated retraction versus hemodynamic loading—A delicate balance in tissue-engineered heart valves. Journal of Biomechanics, 2014. 47(9): p. 2064-2069.
21. Tourniaire, G., et al., Polymer microarrays for cellular adhesion. Chem Commun (Camb), 2006(20): p. 2118-20.
22. Santoro, R., et al., Feasibility of pig and human-derived aortic valve interstitial cells seeding on fixative free decellularized animal pericardium. J Biomed Mater Res B Appl Biomater, 2015.
23. Wendt, D., et al., Oscillating perfusion of cell suspensions through three-dimensional scaffolds enhances cell seeding efficiency and uniformity. Biotechnology and Bioengineering, 2003. 84(2): p. 205-214.
24. Santoro, R., et al., On-line monitoring of oxygen as a non-destructive method to quantify cells in engineered 3D tissue constructs. J Tissue Eng Regen Med, 2012. 6(9): p. 696-701.
25. Roverso, M., et al., A preliminary study on human placental tissue impaired by gestational diabetes: a comparison of gel-based versus gel-free proteomics approaches. Eur J Mass Spectrom (Chichester), 2016. 22(2): p. 71-82.
26. Brioschi, M., et al., A mass spectrometry-based workflow for the proteomic analysis of in vitro cultured cell subsets isolated by means of laser capture microdissection. Analytical and Bioanalytical Chemistry, 2014. 406(12): p. 2817-2825.
27. Van Lieshout, M., et al., A knitted, fibrin-covered polycaprolactone scaffold for tissue engineering of the aortic valve. Tissue Eng, 2006. 12(3): p. 481-7.
28. Sant, S., et al., Effect of biodegradation and de novo matrix synthesis on the mechanical properties of valvular interstitial cell-seeded polyglycerol sebacate-polycaprolactone scaffolds. Acta Biomaterialia, 2013. 9(4): p. 5963-5973.
29. Pilecki, B., et al., Characterization of Microfibrillar-associated Protein 4 (MFAP4) as a Tropoelastin- and Fibrillin-binding Protein Involved in Elastic Fiber Formation. J Biol Chem, 2016. 291(3): p. 1103-14.
30. Wagenseil, J. E. and R. P. Mecham, New insights into elastic fiber assembly. Birth Defects Res C Embryo Today, 2007. 81(4): p. 229-40.

1. (canceled)

2. (canceled)

3. (canceled)

4. The scaffold or a medical device of claim 18 wherein the polymer further comprises a third monomer selected from the group consisting of: BMA, DEGMEMA, DAAA and MMA.

5. The scaffold or a medical device of claim 18 wherein the polymer comprises monomers selected from the group consisting of:
a)-styrene and DMAA,
b)-MMA and GMA or DEAEMA or DMAEA,
c)-MEMA and DEAEA or DEAEMA or BAEMA or 4-vinylpyridine or GMA, and
d)-HEMA and BAEMA or DMVBA or 1-vinylimidazole or 4-vinylpyridine.

6. The scaffold or a medical device of claim 18 wherein the ratio between the first monomer and the second monomer is between 40:60 and 90:10.

7. The scaffold or a medical device of claim 6 wherein the ratio between the first monomer and the second monomer is between 50:50 and 90:10.

8. The scaffold or a medical device of claim 4 wherein the ratio between the first monomer, the second monomer and the third monomer is between 40:30:30 and 60:30:10.

9. The scaffold or a medical device of claim 18 wherein the polymer is functionalized.

10. The scaffold or a medical device of claim 9 wherein the functionalization is carried out by an amine selected from the group consisting of: DnHA, DBnA, TEDETA, Mpi, TMPDA, DEMEDA, TMEDA, Pyrle, MAEPy, BnMA, MnHA, DcHA, cHMA, MAn, DnBA and DnHA.

11. The scaffold or a medical device of claim 18 wherein the polymer is PA6, PA98, PA309, PA316, PA317, PA321, PA338, PA426, PA438 (Ranked with a score 3 according to screening results), PA104, PA111, PA112, PA134, PA167, PA176, PA181, PA187, PA255, PA285, PA295, PA296, PA318, PA319, PA324, PA326, PA329, PA354, PA364, PA506, PA512, PA516, PA531 (Ranked with a score 2 according to screening results) as defined in Table I.

12. (canceled)

13. (canceled)

14. The scaffold or a medical device of claim 18, wherein the medical device is implantable or the scaffold is bio-absorbable.

15. The scaffold or a medical device of claim 14 wherein the medical device is selected from the group consisting of: heart valve substitute, heart valve implant, heart valve bio-artificial tissue, and heart valve tissue scaffold.

16. The scaffold or a medical device of claim 14, wherein the medical device comprises polycaprolactone.

17. (canceled)

18. A scaffold or a medical device coated with or comprising a polymer comprising:
a) a first monomer selected from the group consisting of: styrene, MMA, HEMA and MEMA; and b) a second monomer selected from the group consisting of: GMA, DEAEA, DEAEMA, DMAA, BAEMA, 4-vinylpyridine, DMVBA, 1-vinylimidazole, and DMAEA.

19. The medical device according to claim 18 wherein said device is a tissue engineered heart valve (TEHV) prosthesis.

20. A yarn or a thread manufactured with a polymer comprising:
a) a first monomer selected from the group consisting of: styrene, MMA, HEMA and MEMA; and b) a second monomer selected from the group consisting of: GMA, DEAEA, DEAEMA, DMAA, BAEMA, 4-vinylpyridine, DMVBA, 1-vinylimidazole, and DMAEA.

21. A textile manufactured with the yarn or thread according to claim 20.

22. The scaffold or medical device according to claim 18, further comprising:
a) living cells produced by in vitro incubation and/or
b) additional components selected from the group consisting of growth factors, DNA, RNA, proteins, peptides and therapeutic agents for treatment of disease conditions wherein said cells are attached to the polymer.

23. (canceled)

24. A method to coat a scaffold or a medical device with a polymer comprising:
a) a first monomer selected from the group consisting of: styrene, MMA, HEMA and MEMA; and b) a second monomer selected from the group consisting of: GMA, DEAEA, DEAEMA, DMAA, BAEMA, 4-vinylpyridine, DMVBA, 1-vinylimidazole, and DMAEA comprising coating said scaffold or medical device with said polymer by a method selected from the group consisting of: grafting, dipping, spraying, electrospinning or 3D printing.

25. The textile according to claim 21 manufactured by electrospinning and/or embroidery.

26. A method for repair or replacement of tissue comprising: providing the scaffold or medical device according to claim 18, and locating the said scaffold or medical device on or in the body of a subject.

27. A 3-D printed scaffold manufactured with a polymer comprising:
a) a first monomer selected from the group consisting of: styrene, MMA, HEMA and MEMA; and b) a second monomer selected from the group consisting of: GMA, DEAEA, DEAEMA, DMAA, BAEMA, 4-vinylpyridine, DMVBA, 1-vinylimidazole, and DMAEA.
